(19) United States
(12) Patent Application Publication
(10) Pub. No.: US 2007/0172817 A1
(43) Pub. Date: Jul. 26, 2007

(54) Methods of Producing Antibodies for Diagnostics and Therapeutics

(76) Inventor: Xiao-Jia Chang, Lincoln, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/562,779

(22) Filed: Nov. 22, 2006

Related U.S. Application Data
(63) Continuation of application No. 10/615,343, filed on Jul. 8, 2003, now abandoned.

(51) Int. Cl. C12Q 1/70; G01N 33/53

(52) U.S. Cl. 435/5; 435/7.92

(57) ABSTRACT

Provided herein are methods for making and screening antibodies. Methods may include injecting a plurality of antigens or nucleic acids encoding antigens into a host. Methods may also include using fusion proteins of an antigen to a carrier protein for preparing and/or screening antibody preparations. The methods may be used for simultaneous making and/or screening of large numbers of different antibodies.

### BACKGROUND

Native antibodies are synthesized primarily by specialized lymphocytes called “plasma cells.” Production of a strong antibody response in a host animal is controlled by inducing and regulating the differentiation of B cells into these plasma cells. This differentiation involves virgin B cells (which have a modified antibody as a cell-surface antigen receptor and do not secrete antibodies) becoming activated B cells (which both secrete antibodies and have cell-surface antibodies), then plasma cells (which are highly specialized antibody factories with no surface antigen receptors). This differentiation process is influenced by the presence of antigen and by cellular communication between B cells and helper T cells.

Because of their ability to bind selectively to an antigen of interest, antibodies have been used widely for research, diagnostic and therapeutic applications. The potential uses for antibodies were expanded with the development of monoclonal antibodies. In contrast to polyclonal antiserum, which includes a mixture of antibodies directed against different epitopes, monoclonal antibodies are directed against a single determinant or epitope on the antigen and are homogeneous. Moreover, monoclonal antibodies can be produced in unlimited quantities.

The use of antibody reagents in proteomic research and medical applications is extremely broad and diversified. Such uses range from antibody therapeutics, immunoassays, affinity purification, protein expression, function analysis, tissue and whole body imaging. Antibody microarray technology is currently at its infancy and holds great growth potential in diagnosis and a wide range of other clinical applications. At present however, only a small fraction of the total >100,000 proteins encoded by the whole human genome possess their antibody counterparts. This is mainly due to the fact that current antibody generation is performed on a small scale basis and the process is slow and labor intensive.

For example, in one approach originated by Kohler and Milstein (Kohler and Milstein (1975) Nature 256:495), an antibody-secreting immune cell is first isolated from an immunized mouse and then fused with a myeloma cell, a type of B cell tumor. The resultant hybrid cells (i.e. hybridomas) can then be maintained in vitro. Once established, these hybridomas will continue to secrete antibodies with a defined specificity.

Another approach of producing monoclonal antibodies is phage display library construction. The process proceeds with extraction of mRNA from a repertoire of human peripheral blood cells, followed by construction of a cDNA library comprising sequences of the variable regions of preferably all immunoglobulins. The cDNAs are then inserted into phages to which to display the immunoglobulin variable region as Fab fragments. Theoretically, if the phage library is large enough, it is possible to isolate the particular phage displaying the desired Fab fragment by panning the phages against the antigen of interest. However, this method is generally applicable only to substantially purified antigens, and not to a mixture of antigens such as thousands of those surface antigens expressed on the cell.

Monoclonal antibodies are currently used in clinical trials as therapeutics for both acute and chronic human diseases, including leukemia, lymphomas, solid tumors (e.g., colon, breast, hepatic), AIDS and autoimmune diseases. An example of a commercially available antibody therapeutic agent is anti-Her2 (Trastuzumab or Herceptin). Anti-Her2 is the first humanized antibody approved for the treatment of HER2 positive metastatic breast cancer and is designed to target and block the function of HER2 protein overexpression. Although anti-Her2 has been successful in the treatment of breast cancer, adverse effects of the drug has resulted in 27% of patients developing cardiomyopathy (Horton J.(2002) Cancer Control. 9:499-507, Ewer et al. (2002) Proc Annu Meet Am Soc Clin Oncol. 21 :489). Other adverse effects of this antibody have been reported to include severe hypersensitivity reactions (including anaphylaxis), infusion reactions, and pulmonary events.

Further studies on erbB2, the mouse homolog of Her2, revealed a role for Her2 in the prevention of dilated cardiomyopathy (Crone et al. (2002) Nat Med 8(5):459-465). Another independent clinical study reported myocardial uptake of radiolabeled anti-Her2 in 7 out of 20 patients treated with anti-Her2 (Behr et al. (2002) N Engl J Med. 345:995-996). These studies have led researchers to the conclusion that patients who were receiving anti-Her2 treatment developed cardiomyopathy because anti-Her2 was non-differentially targeting Her2 in breast cancer cells and cardiac cells. The design of anti-Her2 therapeutic antibody did not allow for the antibody to distinguish between mutant Her2 that is overexpressed in diseased tissue and normal Her2 expressed in cardiac tissue. Although the anti-Her2 is highly specific for its target protein, Her2, a significant problem exists in that the antibody is not able to distinguish between diseased tissues and normal, healthy tissues. Accordingly there remains a need for a better designed antibody therapeutic with increased specificity and efficacy.

Thus, there remains a considerable need for a high-throughput process for the production of antibodies for use in diagnostic and therapeutic applications, as well as in drug discovery.

### SUMMARY OF THE INVENTION

Provided herein are methods for identifying an antibody to a target protein from a plurality of antibodies comprising (i) providing antibodies wherein at least one antibody binds specifically to a fusion protein comprising at least a portion of a target protein linked to a carrier protein; (ii) linking at least some of the antibodies to a solid surface to obtain a solid surface coated with antibodies, wherein different antibodies are located on different areas of the solid surface; (iii) contacting the solid surface coated with antibodies with the fusion protein; and (iv) conducting an assay to determine the presence of the carrier protein, wherein the presence of a carrier protein indicates the presence of an antibody to the target protein. The antibodies may be purified or non purified antibody preparations. They may be serum from an immunized or non-immunized animal or they may be hybridoma supernatant.

The target protein may be an isoform of a protein or a portion thereof sufficient for raising an antibody against it. In one embodiment, the isoform of a protein is an isoform that is associated with a disease, e.g., VEGF isoforms VEGF165 and VEGF121, or a portion thereof sufficient for raising an antibody against it. The carrier protein linked to the target protein may comprise of secretory alkaline phosphatase (SEAP), horseradish peroxidase, beta-galactosidase, luciferase, or portions thereof sufficient for enzymatic activity and IgG Fc (gamma chain) or portion thereof The antibodies provided may be linked to a solid surface comprising, e.g., Protein A, Protein A Sepharose, or other Protein A conjugates; Protein G, Protein G Sepharose or other protein G conjugates. Assays to determine the presence of the carrier protein may include a chemiluminescence assay, a fluorescence assay, or a calorimetric assay. Methods for identifying an antibody to a target protein from a plurality of antibodies may further comprise a wash step between steps (iii) and (iv) to remove unbound fusion protein.

Also provided are methods for generating a plurality of monoclonal antibodies, wherein each monoclonal antibody binds to a target protein, comprising (i) administering to a host a plurality of fusion proteins or nucleic acids encoding fusion proteins, wherein each fusion protein comprises at least a portion of a target protein and a carrier protein; (ii) preparing a plurality of monoclonal antibody producing cells obtained from cells from the host; and (iii) screening the cells according to the methods described above, to obtain a plurality of monoclonal antibodies against the target proteins.

The target protein may be an isoform of a protein or a portion thereof sufficient for raising an antibody against it. In one embodiment, the isoform of a protein is an isoform that is associated with a disease, e.g. a viral protein or a portion thereof sufficient for raising an antibody against it. The carrier protein linked to the target protein may comprise of secretory alkaline phosphatase (SEAP), horseradish peroxidase, beta-galactosidase, luciferase, or portions thereof sufficient for enzymatic activity and IgG Fc (gamma chain). A plurality of fusion proteins or nucleic acids encoding fusion proteins, e.g. expression vectors, may be administered to a host, e.g. a mouse. At least 3, 10, 100, or 100 fusion proteins or nucleic acids encoding fusion proteins may be administered at a time to a host.

Also provided herein are methods for generating a plurality of monoclonal antibodies, wherein at least one monoclonal antibody binds to an isoform of a protein that is associated with a disease, comprising (i) administering to a host a plurality of fusion proteins or nucleic acids encoding fusion proteins, wherein each fusion protein comprises at least a portion of an isoform of a protein that is associated with a disease and a carrier protein; (ii) preparing a plurality of monoclonal antibody producing cells from spleen cells obtained from the host; and (iii) screening the cells according to the method of claim 1, to obtain at least one monoclonal antibody that binds to an isoform of a protein that is associated with a disease.

The fusion protein may comprise vascular endothelial growth factor isoform 165 (VEGF165) peptide DRARQENPCGPCSE (SEQ ID NO: 2), or vascular endothelial growth factor isoform 121 (VEGF121) peptide DRARQEKCDKPRR (SEQ ID NO: 4) or HER-2 splice isoform 1peptide INCTHS/PLTS (SEQ ID NO: 6) or HER-2 splice isoform 2 peptide CTHSCV/ASPLT (SEQ ID NO: 8). The carrier protein may comprise of secretory alkaline phosphatase (SEAP), horseradish peroxidase, beta-galactosidase, luciferase, or portions thereof sufficient for enzymatic activity and IgG Fc (gamma chain). A plurality of fusion proteins or nucleic acids encoding fusion proteins, e.g. expression vectors, may be administered to a host, e.g. a mouse. At least 3, 10, 100, or 100 fusion proteins or nucleic acids encoding fusion proteins may be administered at a time to a host, e.g. a mouse.

Provided herein are methods for isolating an antibody binding specifically to a target protein from a plurality of antibodies that are associated with the nucleic acid(s) encoding the antibody, comprising (i) linking at least a portion of a target protein to a pin on a solid surface, which may comprise a plurality of pins, to obtain a pin coated with the protein; (ii) contacting the pin coated with the protein with a plurality of antibodies associated with the nucleic acid(s) encoding the antibody under conditions appropriate for antibody/antigen complexes to form; and (iii) isolating an antibody that is attached to the pin, to thereby isolate an antibody to a target protein.

In one embodiment, the antibodies that are associated with the nucleic acid(s) encoding the antibody are phages. Methods of isolating an antibody may further comprise detaching the antibody from the pin and/or include a wash step between steps (ii) and (iii). The plurality of proteins that are linked to a plurality of pins may comprise different proteins linked to different pins. The solid surface may comprise at least 10, 100, or 1000 pins. A portion of the target protein may be associated with keyhole limpet hemacyanin (KLH), secretory alkaline phosphatase (SEAP), IgG Fc (gamma chain), Glutathione-S-Transferase (GST), or a polyhistidine containing tag. The solid surface may comprise of biotin or streptavidin, nickel, or gluthathione.

Also provided herein are methods for determining the presence of an antigen in a sample, comprising (i) contacting a sample with a solid surface comprising a plurality of antibodies located at specific locations on the solid surface under conditions in which antigen/antibody complexes form specifically; (ii) further contacting the solid surface with a plurality of fusion proteins, wherein each fusion protein comprises a polypeptide that binds specifically to an antibody on the solid surface and a carrier protein, under conditions in which antigen/antibody complexes form specifically; and (iii) detecting the presence of the carrier protein at each specific location on the solid surface, wherein the absence or a reduced amount of the carrier protein at a specific location indicates the presence of antigen binding specifically to the antibody located at the specific location, thereby indicating the presence of the antigen in the sample.

The solid surface may comprise at least about 100 or 1000 antibodies. The solid surface may also be an antibody array, wherein each antibody is located at a specific address on the array. The carrier protein may be an enzyme or a portion thereof sufficient for enzymatic activity and the methods may further comprise contacting the solid surface with a substrate of the enzyme.

Also provided herein are methods of identifying an epitope on a target protein, comprising (i) providing nucleic acids encoding a plurality of fusion proteins, wherein each fusion protein comprises a peptide of 6 to 15 amino acids of the target protein and a carrier protein, and wherein the peptides comprise different sequences of the target protein; (ii) administrating the plurality of fusion proteins to an animal host; (iii) preparing a plurality of monoclonal antibody producing cells obtained from cells from the host; and (iv) screening the cells according to the methods previously described to identify antibodies to the target protein, wherein the presence of an antibody to a peptide indicates that the peptide corresponds to an epitope on the target protein. The peptides may comprise of staggered sequences of the target protein. The protein may be a cell surface receptor and the fusion proteins may further comprise amino acid sequences located in the extracellular domain of the receptor.

Methods for preparing a DNA vaccine against a disease comprising (i) identifying one or more epitopes of a protein associated with the disease according to methods for identifying an epitope on a target protein described herein; and (ii) including nucleotide sequences encoding one or more epitopes into an expression vector, to thereby prepare a DNA vaccine against a disease. Methods for preparing a vaccine against a disease may also comprise(i) identifying one or more epitopes of a protein associated with the disease according to methods for identifying an epitope on a target protein described herein; and (ii) preparing peptides comprising an amino acid sequences of one or more epitopes, to thereby prepare a vaccine against a disease are also provided herein.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1 shows an exemplary method for screening a phage display library.

FIG. 2 shows an exemplary method of epitope scanning.

FIG. 3 shows an exemplary method for screening antibody arrays.

FIG. 4 shows an alignment of VEGF isoforms 121, 165 and 206.

FIG. 5 shows the design of exemplary antibodies to disease associated VEGF isoforms.

FIG. 6 shows the design of exemplary antibodies to disease associated CD44 isoforms.

FIGS. 7A and B show the nucleotide and amino acid sequences of human VEGF (GenBank accession number NM_{—}003376).

FIGS. 8A-G show the nucleotide and amino acid sequences of human ERB2 (GenBank accession number NM_{—}004448).

FIGS. 9A and B show the nucleotide and amino acid sequences of human PSA (GenBank accession number X05332).

### DETAILED DESCRIPTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

As used in the specification and claims, the singular form “a”, “an” and “the” include plural references unless the context clearly dictates otherwise. For example, the term “a cell” includes a plurality of cells, including mixtures thereof.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein the term “antibody” refers to immunoglobulin molecules and antigen-binding portions of immunoglobulin molecules, i.e., molecules that contain an antigen binding site which specifically binds (“immunoreacts with”) an antigen. In an exemplary embodiment, the term “antibody” specifically covers monoclonal antibodies (including agonist, antagonist, and blocking or neutralizing antibodies). Structurally, the simplest naturally occurring antibody (e.g., IgG) comprises four polypeptide chains, two heavy (H) chains and two light (L) chains inter-connected by disulfide bonds. The term “V_{H}” refers to a heavy chain variable region of an antibody. The term “V_{L}” refers to a light chain variable region of an antibody. The natural immunoglobulins represent a large family of molecules that include several types of molecules, such as IgD, IgG, IgA, IgM and IgE. The term also encompasses hybrid antibodies, or altered antibodies, and fragments thereof, including but not limited to Fab fragment(s), and Fv fragment(s). Antibodies can be fragmented using conventional techniques and the fragments screened for utility in the same manner as described for whole antibodies. A Fab fragment of an immunoglobulin molecule is a multimeric protein consisting of the portion of an immunoglobulin molecule containing the immunologically active portions of an immunoglobulin heavy chain and an immunoglobulin light chain covalently coupled together and capable of specifically combining with an antigen. Fab fragments can be prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using any other methods known in the art.

“Antigen” as used herein means a substance to which one would like to raise one or more antibodies. Antigens include but are not limited to peptides, proteins, glycoproteins, polysaccharides and lipids; portions thereof and combinations thereof

An antibody “binds specifically” to an antigen or an epitope of an antigen if the antibody binds preferably to the antigen over most other antigens. For example, the antibody may have less than about 50%, 20%, 10%, 5%, 1% or 0.1% cross-reactivity toward one or more other epitopes.

As used herein, the term “carrier protein” is a protein or peptide that improves the production of antibodies to a protein to which it is associated and/or can be used to detect a protein with which it is associated. Many different carrier proteins can be used for coupling with peptides for immunization purposes. The choice of which carrier to use should be based on immunogenicity, solubility, whether adequate conjugation with the carrier can be achieved and screening assays used to identify antibodies to target proteins. The two most commonly used carriers are keyhole limpet hemocyanin (KLH) and bovine serum albumin (BSA). Other examples include secretory alkaline phosphatase (SEAP), horseradish peroxidase, luciferase, beta-galactosidase, IgG Fc (gamma chain), Glutathione-S-Transferase (GST), polyhistidine containing tags and other enzymes like beta-lactamase, other secretary proteins or peptides.

The terms “isoform of a protein” as used herein refers to polymers of amino acids of any length that are derived from alternative splicing events. Alternative splicing is the process (during transcription) via which alternative exons (i.e., portion of gene that codes-for specific domain of a protein) within a given RNA molecule are combined (by RNA Polymerase molecules) to yield different mRNAs (messenger RNA molecules) from the same gene. Each such mRNA is known as a “gene transcript”. Commonly, a single gene can encode several different mRNA transcripts, caused by cell- or tissue-specific combination of different exons. For example, VEGF165 and VEGF121 are both derived from the VEGF gene. VEGF165 results from deletion of exon 6 (i.e. when Exon 5 and Exon 7 are combined) and VEGF121 results from deletion of exon 6 and 7 (i.e. when Exon 5 and 8 are combined). Other causes/sources of alternative splicing include frameshifting (i.e., different set of triplet codons in the mRNA/transcript is translated by the ribosome) or varying translation start or stop site (on the mRNA during its translation), resulting in a given intron remaining in the mRNA transcript. Different body tissues and some diseases cause alternative splicing (i.e., resulting in different proteins being produced in different tissues; or in diseased tissues) from a given gene.

A “isoform of a protein associated with a disease” refers to any protein or polypeptide derived from an alternative splicing event, whose presence or abnormal level correlates with a disease. For example, it may be found at an abnormal level or in an abnormal form in cells derived from disease-affected tissues as compared with tissues or cells of a non disease control. It may be a protein isoform that is expressed at an abnormally high level; it may be a protein isoform expressed at an abnormally low level, where the altered expression correlates with the occurrence and/or progression of the disease. A disease-associated protein isoform may also be the translated product of a gene possessing mutation(s) or genetic variation that is directly responsible or is in linkage disequilibrium with other gene(s) that are responsible for the etiology of a disease.

The term “epitope” refers to the region of an antigen to which an antibody binds preferentially and specifically. A monoclonal antibody binds preferentially to a single specific epitope of a molecule that can be molecularly defined. An epitope of a particular protein or protein isoform may be constituted by a limited number of amino acid residues, e.g. 5-15 residues, that are either in a linear or non-linear organization on the protein or protein isoform. An epitope that is recognized by the antibody may be, e.g., a short peptide of 5-15 amino acids that spans a junction of two domains or two polypeptide fragments of a disease-associated protein isoform that is not present in the normal isoforms of the protein. A disease-associated protein isoform may be a translation product of an alternatively spliced RNA variant that lacks one or more exon(s) relative to the RNA encoding the normal protein.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product”. If the polynucleotide is derived from genomic DNA, expression may include splicing of the mRNA in a eukaryotic cell.

The term “immunogen” refers to compounds that are used to elicit an immune response in an animal. As used herein, immunogen also refers to fusion proteins and nucleic acids encoding such fusion proteins.

A “monoclonal antibody”, refers to an antibody molecule in a preparation of antibodies, wherein all antibodies have the same specificity and are produced from the same nucleic acid(s). For preparation of monoclonal antibodies directed toward a specific protein, any technique that provides for the production of antibody molecules by continuous cell line culture may be utilized. Such techniques include, but are not limited to, the hybridoma technique (see Kohler & Milstein (1975) Nature 256:495-497); the trioma technique; the human B-cell hybridoma technique (see Kozbor, et al. (1983) Immunol Today 4:72), the EBV hybridoma technique to produce human monoclonal antibodies (see Cole, et al., 1985 In: Monoclonal Antibodies and Cancer Therapy, Alan R. Liss, Inc., pp. 77-96) and phage display. Human monoclonal antibodies may be utilized in the practice of the methods described herein and may be produced by using human hybridomas (see Cote et al. (1983). Proc. Natl. Acad. Sci. USA 80: 2026-2030) or by transforming human B-cells with Epstein Barr Virus in vitro (see Cole et al. (1985) In: Monoclonal Antibodies and Cancer Therapy, Alan R. Liss, Inc., pp. 77-96).

The terms “polynucleotide”, and “nucleic acid” are used interchangeably. They refer to a polymeric form of nucleotides of any length, either deoxyribonucleotides or ribonucleotides, or analogs thereof Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement. An “oligonucleotide” refers to a single stranded polynucleotide having less than about 100 nucleotides, less than about 75, 50, 25, or 10 nucleotides.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “patient”, “subject” or “host” refers to either a human or a non-human animal.

“Target protein” as refers to a protein, e.g., an isoform of a protein, against which one desires to raise an antibody.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

2. Methods for Generating and Screening Antibodies

Provided herein are methods for identifying an antibody that binds to a target protein from a plurality of antibodies, comprising (i) providing antibodies, wherein at least one antibody binds specifically to a fusion protein comprising at least a portion of a target protein linked to a carrier protein; (ii) linking at least some of the antibodies to a solid surface to obtain a solid surface coated with antibodies, wherein different antibodies are located on different solid surfaces or on different locations of one or more solid surfaces; (iii) contacting the solid surface(s) with the fusion protein; and (iv) conducting an assay to determine the presence of the carrier protein, wherein the presence of the carrier protein indicates the presence of an antibody to the target protein. The antibodies may be in purified form, such as immunoglobulin (Ig) preparations, such as serum, e.g., polyclonal antiserum of immunized animals; monoclonal antibodies; cultured cell medium, such as hybridoma supernatant; or they may be ascites of experimental animals. Alternatively, antibodies and fusion proteins are first contacted together prior to contacting them with a solid surface. The method may also comprise, first generating monoclonal antibodies to fusion proteins, e.g., by administering to a host a fusion protein and preparing antibody producing cells from cells obtained from the host. In one embodiment, generating monoclonal antibodies comprises (i) administering to a host a plurality of fusion proteins or nucleic acids encoding fusion proteins, wherein each fusion protein comprises at least a portion of a target protein and a carrier protein; (ii) preparing a plurality of monoclonal antibody producing cells, e.g., hybridomas, from cells obtained from the host; and (iii) screening the monoclonal antibody producing cells to isolate those of the desired specificity, such as by detecting the carrier protein. For example, at least 3, 10, 100, 300 or 1000 fusion proteins or nucleic acids encoding fusion proteins may be administered to a host. Screening antibody producing cells for those producing antibodies to each of the fusion proteins is facilitated by using the screening assay described herein, e.g., in which the presence of a desired antibody is detected by detection of the carrier protein after binding of the antibodies to fusion proteins. In one embodiment, the carrier protein is the same or essentially the same for all of the fusion proteins administered to a host. In the latter embodiment, screening is particularly easy, since the same assay will allow identification of cells producing numerous different antibodies.

Persons of skill in the art will recognize that antibodies may also be made against target proteins that are not linked to a carrier protein, and the antibody producing cells are screened with a fusion protein comprising at least a portion of a target protein and a carrier protein. Accordingly, in some embodiments, the protein that is administered to a host is different from the protein that is used for screening antibody producing cells. Of course, even if a protein that is administered to a host does not comprise an amino acid sequence of a carrier protein that is used for detecting antibodies, the protein may nevertheless comprise an amino acid sequence of a protein or peptide for enhancing the immune reaction in the host.

In some embodiments, antibodies are obtained by administrating to a host a plurality of proteins, e.g., fusion proteins. In other embodiments, antibodies are obtained by administrating to a host one or more nucleic acids encoding a plurality of proteins, e.g., fusion proteins. For example, a single nucleic acid encoding a plurality of proteins can be administered to a host for preparing antibodies to the plurality of proteins. Alternatively, two or more nucleic acids encoding two or more proteins are administered to a host for preparing antibodies to two or more proteins. When using one nucleic acid for encoding two or more proteins, the nucleic acid may comprise two or more promoters and/or other regulatory elements. The nucleic acid may also comprise several ribosome binding sites between the open reading frames encoding the two or more proteins.

In one embodiment, the carrier protein is a protein that facilitates the identification of an antibody to a target protein from a plurality of antibodies. Carrier proteins may be detected by a variety of methods. The appropriate method may depend on the type of carrier protein. For example, a carrier protein can be detected using an antibody binding specifically to the carrier protein. Accordingly, a carrier protein may be any protein or molecule to which an antibody is available or can be prepared. For example, a carrier protein may be a tag, such as a histidine tag. A carrier protein may be the constant region of an immunoglobulin molecule, e.g., IgG Fc. Carrier proteins can also be proteins or other molecules that are labeled, e.g., with a fluorescent, phosphorescent or radioactive label. Yet other carrier proteins may be enzymes or portions thereof sufficient for enzymatic activity. For example, a carrier protein can be secretory alkaline phosphatase (SEAP), horseradish peroxidase, luciferase, beta-galactosidase or portions thereof sufficient for enzymatic activity. The enzymes may be of any desired species, e.g., human or non-human such as mouse.

Also provided herein are methods for preparing antibodies to disease associated proteins or disease associated isoforms of proteins. In one embodiment, a human target gene sequence is chosen from a database and oligonucleotides encoding about 10-15 amino acids of the target sequence are included in an expression vector, in phase with a carrier protein. The nucleic acids are then introduced into an animal, e.g., a mouse, for in vivo expression of antigen and for stimulation of an immune response. B cells are then isolated from the animal and hybridomas are produced. Hybridomas are then screened according to methods described herein, e.g., by the detection of the carrier protein.

Proteins, e.g., fusion proteins, may be prepared by chemical synthesis according to methods of protein synthesis known in the art. Proteins can also be made recombinantly. In particular, fusion proteins may be generated by fusing a nucleic acid encoding a target protein or a portion thereof and a nucleic acid encoding a carrier protein or a portion thereof

Nucleic acids encoding target proteins and carrier proteins may be obtained by e.g., polymerase chain reaction (PCR), amplification of gene segments from genomic DNA, cDNA, RNA (e.g. by RT-PCR), or cloned sequences. PCR primers are chosen, based on the known sequences of the genes or cDNA, so that they result in the amplification of relatively unique fragments. Computer programs may be used in the design of primers with required specificity and optimal amplification purposes. See e.g., Oligo version 5.0 (National Biosciences). Factors which apply to the design and selection of pimers for amplification are described for example, by Rylchik, W. (1993) “Selection of Primers for Polymerase Chain Reaction.” In Methods in Molecular Biology, vol. 15, White B. ed., Humana Press, Totowa, N.J. Sequences may be obtained from GenBank or other public sources. Alternatively, the nucleic acids of this invention may also be synthesized by standard methods known in the art, e.g. by use of an automated DNA synthesizer (such synthesizers are commercially available from Biosearch, Applied Biosystems, etc).

Suitable cloning vectors for expressing a protein in a host or in a cell may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, or may carry genes for a marker that can be used in selecting clones containing the vector. Suitable examples include, but are not limited to, plasmids and bacterial viruses, e.g., pUC18, mp18, mp19, pBR322, pMB9, ColE 1, pCR1, RP4, phage DNAs, and shuttle vectors such as pSA3 and pAT28. These and many other cloning vectors are available from commercial vendors such as BioRad, Stratagene, and Invitrogen.

Expression vectors for use in the methods described herein generally are replicable polynucleotide constructs that contain a polynucleotide encoding the target protein of interest or a portion thereof, linked to a carrier protein or a portion thereof, if applicable. The polynucleotide as described herein is operatively linked to suitable transcriptional controlling elements, such as promoters, enhancers and terminators. For expression (i.e., translation), one or more translational controlling elements are also usually required, such as ribosome binding sites, translation initiation sites, and stop codons. These controlling elements (transcriptional and translational) may be derived from the target protein of interest, or they may be heterologous (i.e., derived from other genes or other organisms). A polynucleotide sequence encoding a signal peptide can also be included to allow the polypeptide to cross or lodge in cell membranes or be secreted from the cell. A number of expression vectors suitable for expression in eukaryotic cells including yeast, avian, and mammalian cells are known in the art. One example of an expression vector is pcDNA3 (Invitrogen, San Diego, Calif.), in which transcription is driven by the cytomegalovirus (CMV) early promoter/enhancer. This vector also contains recognition sites for multiple restriction enzymes for insertion of the polynucleotide of interest. Suitable cloning and expression vectors include any known in the art, e.g., those for use in bacterial, mammalian, yeast and insect expression systems. Specific vectors and suitable host cells are known in the art and need not be described in detail herein. For example, see Gacesa and Ramji (1994) Vectors, John Wiley & Sons.

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will grow under selective conditions. Typical selection genes either: (a) confer resistance to antibiotics or other toxic substances, e.g., ampicillin, neomycin, methotrexate; (b) complement auxotrophic deficiencies; or (c) supply critical nutrients not available from complex media. The choice of the proper marker gene will depend on the host cell, and appropriate genes for different hosts are known in the art. Cloning and expression vectors typically contain a replication system recognized by the host.

Expression vectors for expressing proteins in host animals can be, e.g., virus based vectors.

Where a protein is administered to a host animal, both eukaryotic and prokaryotic host systems can be used for producing the protein recombinantly. The polypeptide may then isolated from lysed cells or from the culture medium and purified to the extent needed for its intended use. Examples of prokaryotic host cells appropriate for use with this invention include Escherichia coli. Examples of eukaryotic host cells include avian, insect, plant, and animal cells such as COS7, HeLa, CHO cells and myeloma cells. Mammalian cell lines are also often used as host cells for the expression of polypeptides derived from eukaryotes. Propagation of mammalian cells in culture is well known. See Tissue Culture, Academic Press, Kruse and Patterson, eds. (1973).

“Transformation” refers to the introduction of vectors containing the nucleic acids of interest directly into host cells by well known methods. Transformation methods, which vary depending on the type of host cell, include electroporation; transfection employing calcium chloride, rubidium chloride calcium phosphate, DEAE-dextran, or other substances; microprojectile bombardment; lipofection; infection (where the vector is an infectious agent); and other methods. See generally, Sambrook et al. (1989) and Ausubel et al., (ed.), (1987). Reference to cells into which the nucleic acids described above have been introduced is meant to also include the progeny of such cells.

Once introduced into a suitable host cell, for example, E. coli or COS-7, expression of a fusion protein can be determined using any of the assays described herein. For example, presence of a polypeptide can be detected by chemiluminescent, fluorescence, or calorimetric assays of culture supernatant or cell lysates based on the identity of the carrier protein within the fusion protein.

Certain polypeptides which are fragments of the whole molecule may alternatively be prepared from enzymatic cleavage of intact polypeptides. Examples of proteolytic enzymes include, but are not limited to, trypsin, chymotrypsin, pepsin, papain, V8 protease, subtilisin, plasmin, and thrombin. Intact polypeptides can be incubated with one or more proteinases simultaneously or sequentially. Alternatively, or in addition, intact polypeptides can be treated with disulfide reducing agents. Peptides may then be separated from each other by techniques known in the art, including but not limited to, gel filtration chromatography, gel electrophoresis, and reverse-phase HPLC.

Preparation of antibodies may be accomplished by any number of well-known methods for generating antibodies, e.g., monoclonal antibodies. Methods for making monoclonal antibodies typically include a step of injecting a host, typically a mouse, with the desired immunogen. In one embodiment, a plurality of proteins, e.g., fusion proteins, is injected, wherein each fusion protein comprises at least a portion of a target protein and a carrier protein. In another embodiment, a plurality of nucleic acids encoding proteins, e.g., fusion proteins, is injected, wherein each fusion protein comprises at least a portion of a target protein and a carrier protein. In a particular embodiment, the host is a rodent, e.g. a mouse. The mouse to be immunized may, for example, be an “antigen-free” mouse as described in U.S. Pat. No. 5,721,122.

In one embodiment, the host is a transgenic animal in which human immunoglobulin loci have been introduced. For example, the transgenic animal may be a mouse comprising introduced human immunoglobulin genes and one in which the endogenous immunoglobulin genes have been partially or completely inactivated. Upon challenge, human antibody production in such transgenic hosts is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following publications: Marks et al., Bio/Technology 10: 779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg and Huszar, Intern. Rev. Immunol. 13:65-93 (1995).

The host animal may be immunized with the antigens in a variety of different ways. For example, by subcutaneous, intramuscular, intradermal, intravenous, and/or intraperitoneal injections. In addition, injections into lymphoid organs, popliteal lymph node and/or footpads are possible. It may be desirable to immunize the animal using a combination of two or more different administration routes, separately and/or simultaneously.

The amount of each fusion protein administered to the host animal may, for example, range from about 0.01 μg to about 250 μg, preferably from about 1 μg to about 100 μg. Alternatively, the amount of nucleic acids encoding a plurality of fusion proteins administered to the host animal may, for example range from 0.01 micrograms to about 100 μg, preferably from about 1 μg to 25 μg. For example, a mouse may be injected with 10 μg of protein or 10 μg of nucleic acid.

In certain embodiments, a host animal is injected with three or more different proteins, such as fusion proteins, or nucleic acids encoding such, e.g., at least about 3, 10, 30, 100, 300, or 1000 different proteins or nucleic acids encoding proteins or combinations thereof In one embodiment of the invention, a host animal is injected with a composition comprising a mixture of the two or more different proteins or nucleic acids encoding proteins and, optionally, a physiologically acceptable diluent, such as PBS or other buffer. Alternatively, a host animal is injected sequentially with proteins or nucleic acids encoding the proteins. The fusion proteins used to prepare the composition have preferably been purified by at least by one purification step.

The methods described herein allow the production of antibodies with defined epitope specificities. Antigens for preparing antibodies are preferably at least the minimum number of amino acids that are recognized by antibodies, e.g., at least 6 amino acids long. Antigens may also be at least 10 amino acids, at least about 15, 20, 50, or 100 amino acids long. Accordingly, antigens may be from 6 to 15 amino acids long. In addition to antibodies to linear epitopes (contiguous amino acids), antibodies to three dimensional epitopes, i.e., non linear epitopes, can also be prepared, based on, e.g., crystallographic data of proteins. Hosts may be injected with polypeptides of overlapping sequence across a desired area of a protein. For example, short antigens (or peptide antigens) may be designed in tandem order of linear amino acid sequence of a protein, or staggered in linear sequence of the protein. Hosts may also be injected with peptides of different lengths encompassing a desired target sequence. At least one antibody from a plurality of antibodies is expected to bind to the full length or partially native protein. It is also expected that antibodies blocking biological functions or neutralizing antibodies will be identified using the methods described herein.

A plurality of short antigens, e.g., the length of an epitope, can be designed for one target protein and administered to one host. Alternatively, a plurality of short antigens having sequences from different proteins can be administered to one host.

In one embodiment, protein isoform specific antibodies are generated. Such antibodies may be directed to short peptidic sequences that are located (i) within an exon of a particular protein isoform; (ii) across an exon-exon border of a particular isoform; or (iii) spanning a deletion site in one exon. Such short sequences are referred to as “signature epitopes,” since it is specific to a particular isoform. A “signature epitope” can also be a three-dimensional epitope formed by non-linear amino acid sequences. It may, e.g., represent a three dimensional epitope that represents a conformational feature of the target protein isoform that is not present in other isoforms of the target protein.

At different time points following the injection of a plurality of nucleic acids encoding proteins of interest, the successful production of proteins from said nucleic acids may be measured from the serum of injected host animals. Assays used in the measurement may depend on whether it is linked to a carrier protein and if so, what the carrier protein is. In one embodiment, the carrier protein is secretory alkaline phosphatase (SEAP). The measurement of the production of SEAP fusion proteins or others may involve obtaining a sample of blood from the saphenous veins of the injected mouse and diluting the serum sample with saline solution. The levels of SEAP fusion proteins may then be measured using an assay that allows the measurement of a signal that is emitted following the addition of alkaline phosphatase substrate. Commercially available assays utilizing SEAP includes but is not limited to Clontech's Great EscAPe™ SEAP Assay. Assays for other fusion proteins include but are not limited to commercially available assays utilizing colorimetric, fluorogenic or chemiluminescent substrates for galactosidase, HRP, lactamase and lusiferase. These assays are adaptable to high throughput screening of antibodies.

Where the primary response is weak, it may be desirable to boost the animal at spaced intervals until the antibody titer increases or plateaus. After immunization, samples of serum (test bleeds) may be taken to check the production of specific antibodies. Preferably, the host animal is given a final boost about 3-5 days prior to isolation of immune cells from the host animal.

Antibodies obtained from that injection may be screened against the short antigens of one target protein or against various target proteins. Antibodies prepared against a peptide may be tested for activity against that peptide as well as the native target protein. Antibodies may have affinities of at least about 10⁻⁶M, 10⁻⁷M, 10⁻⁸M, 10⁻⁹M, 10⁻¹⁰M, 10⁻¹¹M or 10⁻¹²M toward the peptide and/or the native target protein.

Monoclonal antibodies may be made using the hybridoma method first described by Kohler et al., Nature, 256:495 (1975). In the hybridoma method, spleenocytes that produce or are capable of producing antibodies are obtained from the animal immunized as described above. Such cells may then be fused with myeloma cells using a suitable “fusing agent”, such as polyethylene glycol or Sendai virus, to form a hybridoma cell [Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and P3X63AgU.1, SP-2 or X63-Ag8-653 cells available from the American Type Culture Collection, Manassas, Va., USA. The 210-RCY3.Ag1.2.3 rat myeloma cell line is also available. Human myeloma and mouse-human heteromyeloma cell lines also have also been described for the production of human monoclonal antibodies [Kozbor, J. Immunol., 133:3001 (1984); Brodeur et al., Monoclonal Antibody Production Techniques and Applications, pp. 51-63 (Marcel Dekker, Inc., New York, 1987)].

Alternatively, hybridoma cell lines may be prepared from the immune cells of the immunized animal in other ways, e.g. by immortalizing the immune cells with a virus (e.g. with Epstein Barr Virus), or with an oncogene in order to produce an immortalized cell line producing the monoclonal antibody of interest. See, also, Babcook et al. PNAS (USA), 93:7843-7848 (1996), concerning production of monoclonal antibodies by cloning immunoglobulin cDNAs from single cells producing specific antibodies for yet another strategy for preparing monoclonal antibodies using immune cells of the immunized animal.

Cells producing antibodies are then screened to identify those producing antibodies to the desired protein. Generally, antibody screens for those which bind to each antigen with which the animal has been immunized may be performed on culture supernatant and/or purified antibodies, e.g., from each hybridoma culture supernatant resulting from fusion as described herein.

In one embodiment, monoclonal antibodies to be tested may be bound to a solid phase e.g., a solid phase comprising Protein A, Protein A Sepharose, or other protein A conjugates, Protein G, Protein G Sepharose or other protein G conjugates. Antibody producing cells are usually screened in multiwell plates. The solid surface is then contacted with antigen. Alternatively, the antibody-antigen complex may be allowed to form by immunoprecipitation prior to binding of the monoclonal antibody to be tested to a solid phase. Once the antibody-antigen complexes are bound to the solid phase, unbound antigen may be removed by washing and positives may be identified by detecting the antigen.

In one embodiment, the antigen comprises a carrier protein. In such embodiments, the presence of an antigen bound to an antibody may be detected by an agent that detects the carrier protein. For example, a carrier protein may be detected by a method using an agent that specifically binds to the carrier protein, such as an antibody. If the carrier protein is an enzyme or portion thereof sufficient for enzymatic activity, the carrier protein may be detected by an enzymatic assay. Accordingly, chemiluminescence assays, fluorescence assays, or calorimetric assays may be conducted pursuant to methods known in the art.

After hybridoma cells that produce antibodies of the desired specificity, affinity, and/or activity are identified, single-cell clones may be subcloned by limiting dilution procedures [Goding, Monoclonal Antibodies: Principles and Practice, pp. 59-103 (Academic Press, 1986)]; single cell cloning by picks; or cloning by growth in soft agar [Harlow and Lane, Antibodies: A Laboratory Manual Cold Spring Harbor Laboratory (1988); pps 224-227].

Hybridoma clones may be grown by standard methods. Suitable culture media for this purpose include, for example, DMEM or RPMI-1640 medium. In addition, hybridoma cells may be grown in vivo as ascites tumors in an animal. [Harlow and Lane, Antibodies: A Laboratory Manual Cold Spring Harbor Laboratory (1988); Chapter 7].

The monoclonal antibodies secreted by the subclones may be suitably separated from the culture medium, ascites fluid, or serum by conventional immunoglobulin purification procedures such as, for example, protein G or A-Sepharose, hydroxylapatite chromatography, gel electrophoresis, dialysis, or affinity chromatography.

Following the isolation of antibodies against desired antigens, the antibodies can further be manipulated or modified. In one embodiment, chimeric antibodies are produced. “Chimeric” antibodies are encoded by immunoglobulin genes that have been genetically engineered so that the light and heavy chain genes are composed of immunoglobulin gene segments belonging to different species. For example, the variable (V) segments of the genes from a mouse monoclonal antibody, e.g., as obtained as described herein, may be joined to human constant (C) segments. Such a chimeric antibody is likely to be less antigenic to a human than antibodies with murine constant regions as well as murine variable regions.

As used herein, the term humanized antibody (HuAb) refers to a chimeric antibody with a framework substantially identical (i.e., at least 85%) to a human framework, having CDRs from a non-human antibody, and in which any constant region present has at least about 85-90%, and preferably about 95% polypeptide sequence identity to a human immunoglobulin constant region. See, for example, PCT Publication WO 90/07861 and European Patent No. 0451216. Hence, all parts of such a HuAb, except possibly the CDR's, are substantially identical to corresponding parts of one or more native human immunoglobulin sequences. The term “framework region”, as used herein, refers to those portions of immunoglobulin light and heavy chain variable regions that are relatively conserved (ie., other than the CDR's) among different immunoglobulins in a single species, as defined by Kabat, et al. (1987) Sequences of Proteins of Immunologic Interest, 4^{th} Ed., US Dept. Health and Human Services.

Human constant region DNA sequences can be isolated in accordance with well known procedures from a variety of human cells, but preferably from immortalized B cells. The variable regions or CDRs for producing humanized antibodies may be derived from monoclonal antibodies capable of binding to the antigen, and will be produced in any convenient mammalian source, including, mice, rats, rabbits, or other vertebrates capable of producing antibodies, by well known methods.

Suitable cells for the DNA sequences and host cells for antibody expression and secretion can be obtained from a number of sources, such as the American Type Culture Collection (“Catalogue of Cell Lines and Hybridomas” 5^{th} edition (1985) Rockville, Md., U.S.A.). Aside from the methods described above for obtaining antibodies (by immunizing a host with one or more antigens), other techniques are available for generating antibodies. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)].

In one embodiment, a library of antibodies, wherein each antibody is associated with the nucleic acid(s) encoding the antibody, such as a phage display library, is used in a high throughput screen for antibodies to one or more antigens. In a particular embodiment, one or more antigens, e.g., portions of target proteins, are linked to one or more pins or extensions of a solid surface, wherein different antigens are linked to different pins. Solid surfaces may have a plurality of pins, e.g., at least 2, 5, 10, 25, 50, 100, 300, 1000 or 3000 pins. Solid surfaces with a plurality of pins are referred to as “multi-pin surfaces.” A solid surface can have as many pins as wells in multiwell plates. Exemplary solid surfaces with pins are those that are made to fit into dishes, e.g., multiwell plates. Solid surfaces with pins are commercially available, e.g., from Nelge NUNC or V&P Scientific, Inc., or can be made. Proteins and fusion proteins can be prepared synthetically or recombinantly, e.g., by expression in COS cells. Binding of proteins to solid surfaces can be conducted by methods known in the art. For example, solid surfaces can be coated with avidin, streptavidin, nickel, glutamine, anti-Flag antibody or anti-human Fc antibody. The solid surface may then be contacted with a library of antibodies, wherein each antibody is associated with nucleic acid(s) encoding the antibody, e.g., a phage display library, under conditions in which antibodies bind specifically to particular antigens. Contacting is done for a time sufficient for antigen-antibody complex formation to occur. The solid surface may then be washed to remove unbound antibodies, and the solid surface is placed above a multiwell dish such that essentially each pin or extension is positioned in a different well of the dish. The antigens or antigen/antibody complexes can then be separated from the solid surface (eluted), such as by an acidic wash, as known in the art, and the antigens or antigen/antibody complexes can be recovered in the wells of a multiwell dish. More antibody can then be produced from the nucleic acid that is associated with the antibody, e.g., from the phage. This process can be repeated several times. A multiwell dish may have at least 12, 24, 48, 96, 384 or 1536 wells wells. Other solid surfaces that can be used include multiwell dishes and beads (e.g., Dynabeads®), wherein, e.g., antigens are in different wells or on different beads. Solid surfaces designed for this purpose and optionally having antigens linked to them are encompassed herein. Antigens for use in these methods may consist of proteins that are linked or not linked to a carrier protein. In embodiments in which a carrier protein is associated with the antigen, the detection of an antibody/antigen complex can be conducted with assays detecting the presence of the carrier protein. Alternatively, the carrier protein can be used to link the antigen to a solid surface. When using a carrier protein, antibodies reacting only to the carrier protein can be eliminated, e.g., by passing the library on a solid surface precoated with carrier protein.

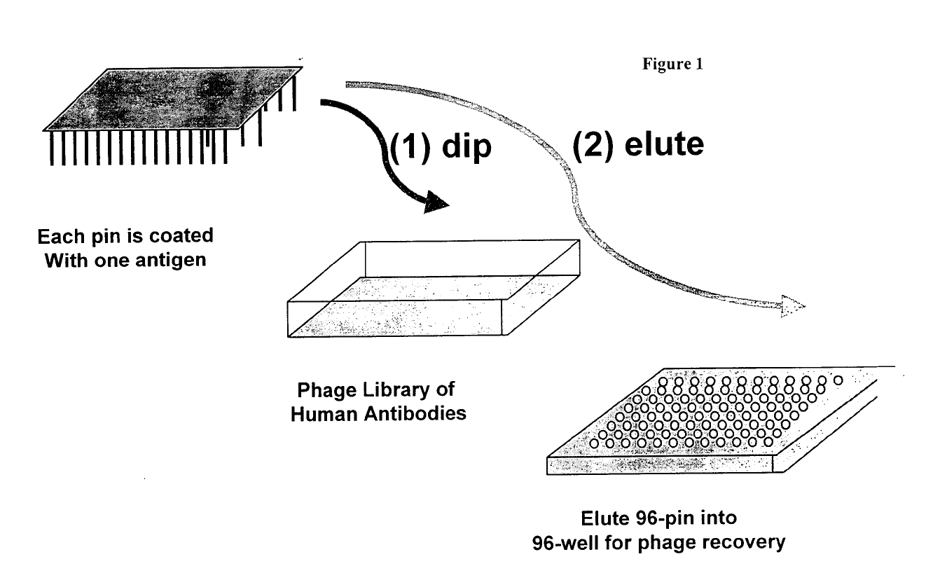

One embodiment of a method comprising screening phage display libraries is set forth in FIG. 1.

Antibody libraries, e.g., phage display libraries, can be produced from the nucleic acids isolated from a naïve human repertoire or from a disease oriented repertoire, e.g,. cancer patients. Phage display libraries are further described in Hoogenboom and Winter, J. Mol. Biol., 227:381 (1992); Marks et al., J. Mol. Biol., 222:581 (1991). Suitable methods for preparing phage libraries have been reviewed and are described in Winter et al., Annu. Rev. Immunol., 12:433-55 (1994); Soderlind et al., Immunological Reviews, 130:109-123 (1992); Hoogenboom, Tibtech February 1997, Vol. 15; Neri et al., Cell Biophysics, 27:47-61 (1995). Libraries of single chain antibodies may also be prepared by the methods described in WO 92/01047, WO 92/20791, WO 93/06213, WO 93/11236, WO 93/19172, WO 95/01438 and WO 95/15388. Antibody libraries are also commercially available, for example, from Cambridge Antibody Technologies (C.A.T.), Cambridge, UK.

Methods of antibody purification are well known in the art. See, for example, Harlow and Lane (1988) Antibodies: A Laboratory Manual, Cold Spring Harbor Laboratory, N.Y. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-antibody. Antibodies may also be purified on affinity columns according to methods known in the art.

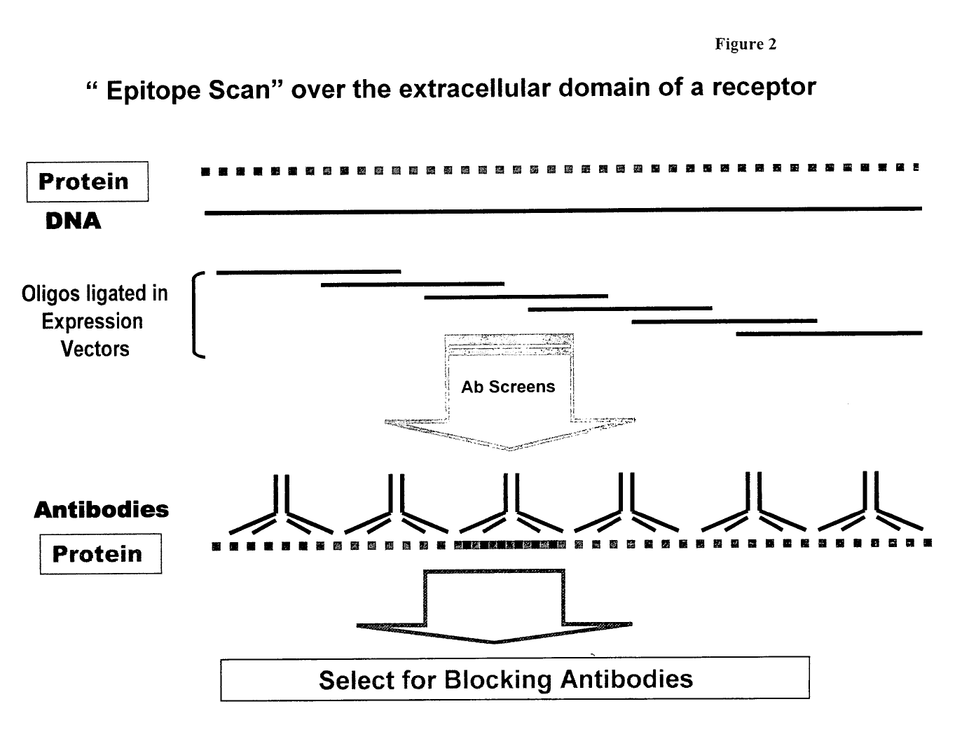

The methods described herein can be used for “epitope scanning” (see, FIG. 2, as an exemplary method). In one embodiment, oligonucleotides having short overlapping and staggered sequences of a particular target protein are included in an expression vector for producing proteins, such as fusion proteins. The expression vectors can then be administered to a host for the production of antibodies, and the epitopes bound by the antibodies produced are identified. For example, serum from the immunized host may be contacted with the fusion proteins and the amount of antibody to each fusion protein determined. In certain embodiments, oligonucleotides encoding peptides or peptides are administered to a host and fusion proteins comprising the peptides and a carrier protein are used for screening the serum. Methods may also comprise preparing monoclonal antibodies from the immunized host and screening the monoclonal antibodies with fusion proteins comprising a carrier protein.

The short overlapping amino acid sequences may also be chemically synthesized and conjugated to a carrier protein. These fusion proteins may then be used to coat the solid surface of multi-pin plates and subject to contacting with an antibody library, e.g. phage display library. Antibodies to each epitope of the scanned region will be isolated from the antibody library and tested for neutralizing, or “blocking function” activities.

Antibodies can then be used, e.g., as blocking antibodies. In addition, a comparison of the sequences to which antibodies were obtained and those to which no antibody was obtained will indicate the location of epitopes in the target protein. The knowledge of the location of epitopes in proteins can be used for the generation of therapeutics, e.g., small molecules. This can be applied, e.g., to determine the location of epitopes in the extracellular domain of receptors, such as G protein coupled receptors (GPCRs). This method can be used to obtain blocking antibodies against GPCRs including chemokine and hormone receptors.

In one embodiment, a method for identifying an epitope on a target protein comprises (i) providing nucleic acids encoding a plurality of fusion proteins, wherein each fusion protein comprises a peptide of 6 to 15 amino acids of the target protein and a carrier protein, and wherein the peptides comprise different sequences of the target protein; (ii) administrating the plurality of fusion proteins to an animal host; (iii) obtaining serum from the animal host; and screening the serum to identify or quantify antibodies to epitopes of the target protein, wherein the presence of an antibody to a peptide indicates that the peptide corresponds to an epitope on the target protein. In another embodiment, the method comprises steps (i) and (ii) above; (iii) preparing a plurality of monoclonal antibody producing cells obtained from cells from the host; and (iv) screening the cells according to the methods described herein to identify antibodies to the target protein, wherein the presence of an antibody to a peptide indicates that the peptide corresponds to an epitope on the target protein. The peptides may comprise staggered sequences of the target protein.

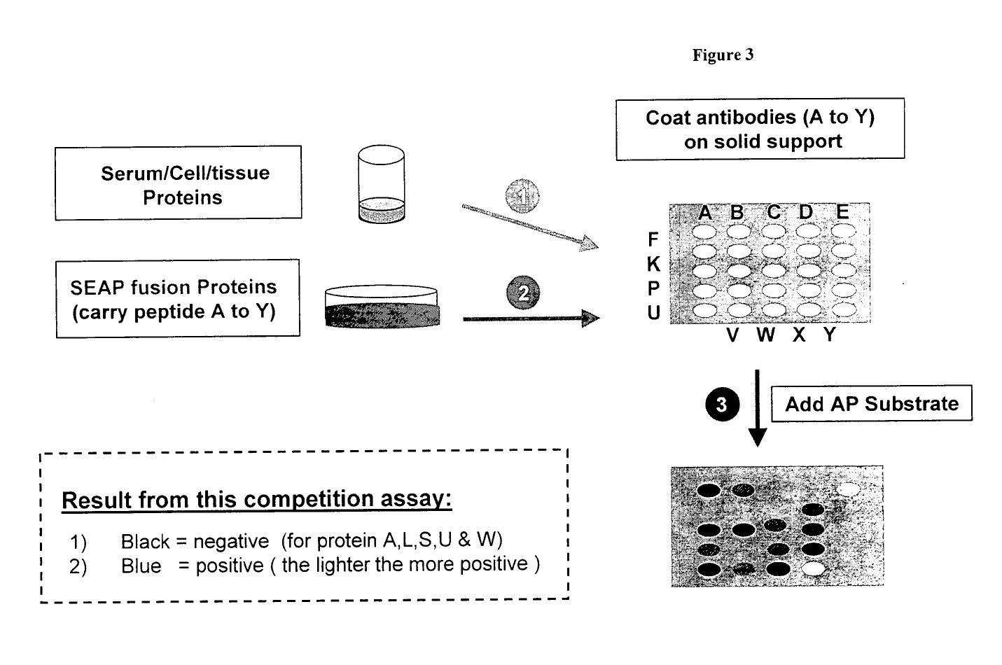

The methods, e.g., assays for detection of antigen and antibody binding, described herein can further be used for screening antibody arrays. In one embodiment, an antibody array is incubated with test proteins, e.g., serum, cell or tissue proteins, under conditions in which antibody/antigen complexes are capable of forming (see, e.g., FIG. 3). The non-binding proteins are washed away. The array is then contacted with fusion proteins comprising peptides, e.g., peptides that bind to each of the antibodies on the array, linked to a carrier protein, e.g., SEAP. After washing unbound protein, the carrier protein is detected, e.g., by adding an alkaline phosphatase substrate, and the array is read. A location on the array that is read as positive will indicate that no protein inhibited binding of the peptide-carrier protein that was added, and therefore that the sample tested did not contain a protein that is recognized by the particular antibody. Accordingly, the less carrier protein that is detected with a particular antibody, the more protein recognized by the antibody was present in the sample.

3. Therapeutic Uses

Antibodies obtained as described herein may be used for treating or preventing diseases in which the presence of an antibody to a particular molecule is beneficiary. In one embodiment, antibodies are used for targeting agents, such as toxins, to particular cells. For example, cancer cells can be killed by delivering a toxin to the cancer cell using an antibody that specifically binds to a protein on the surface of the cancer cell. In a preferred embodiment, such targeting antibodies do not bind to proteins that are present on normal cells. For example, one may use antibodies that bind specifically to disease-associated isoforms, or splice variants, of a protein, i.e., an isoform of a protein that is present essentially only in or on diseased, e.g., cancerous, cells. Of course, if the isoform appears on a normal tissue that is located at a different site in the body, targeting that isoform may be possible, provided that the targeting antibody does not kill all the cells of the normal tissue.

In addition to targeting sequences in exons of disease-associated isoforms, antibodies may also be targeted to exon-exon junctions that are not found in iso forms of proteins that are not associated with disease. Antibodies may also be targeted to three dimensional epitopes that are associated with disease, e.g., not found in normal isoforms of a protein. In addition to antibodies to linear epitopes (continguous amino acids), antibodies to three dimensional epitopes, i.e., non linear epitopes can also be prepared, based on, e.g., crystallographic data of protein isoforms.

In certain embodiments, a plurality of antibodies are administered to a subject. The antibodies can be different antibodies directed to the same antigen, e.g., to a different epitope of the antigen, or they can be directed to different antigens. Certain treatments will comprise a combination of both schemes. These various antibodies can be prepared simultaneously according to methods described herein. For example, a plurality of peptides that are specific to a disease associated form of a protein or nucleic acid(s) encoding such can be injected into a host animal for the preparation of monoclonal antibodies.

Also provided herein are DNA vaccines comprising a nucleotide sequence encoding an epitope of a disease associated protein isoform, which may be used for the prevention or treatment of diseases such as cancers. The epitope may be a short peptide of 10-15 amino acid residues from a linear or non-linear sequence of a disease associated protein isoform. The epitope may span a junction site between two exons, which junction is unique to the particular protein isoform that is associated with a disease and not present in the protein isoform that is found in normal subjects or in normal tissues of diseases subjects. In certain embodiments, DNA vaccines will encode two or more epitopes from a single protein isoform or from multiple protein isoforms and may be used in such combination, e.g., for certain disease indications. DNA vaccines may also encode an epitope specific sequence, e.g., encoding 10-15 amino acids, fused in frame to a carrier protein such as serum albumin, SEAP or other secreted peptide or protein. DNA vaccines may be used for preventing or treated diseases as further described herein. Exemplary DNA vaccines comprise nucleotide sequences encoding peptides described herein, or identified as described herein.

Protein isoforms that have been demonstrated to be associated with disease may be identified through databases such as
PubMed (http://www.ncbi.nlm.nih.gov/PubMed/),
PubMed Central (http://www.pubmedcentral.nih.gov/about/intro.html),
OMIM (http://www.ncbi.nlm.nih.gov/entrez/query.fcgi?db=OMIM),
PROW (http://www.ncbi.nlm.nih.gov/PROW/).

Once identified, sequences of disease associated isoforms may be retrieved from the GenBank database (http://www.ncbi.nlm.nih.gov/Genbank/GenbankSearch.html). Table 1 presents examples of proteins having isoforms associated with disease(s) and the GenBank Accession Numbers of the isoforms.

| | | Spliced variant/ | |
|---|---|---|---|
| Gene | Accession No. | Protein isoform | Disease Association |
| P53 | NM_000546 | Many spliced variants and | Many types of cancers |
| | NP_000537.2 | mutations | |
| Fas | M67454 | Deletion of | Silicosis |
| | AAA63174.1 | transmembrane domain, | |
| | | soluble in serum | |
| Factor H | M17517 | Spliced short variant | ovarian cancer; |
| | AAA52016.1 | | spliced Factor H is 5.5- |
| | | | fold higher in cancer |
| | | | than in normal tissue |
| CD86 | BC040261 | CD86 (deltaTM) | Leukemia, AML |
| | AAH40261.1 | | |
| erb-B2/Her2 | M11730 | HER2-splice (deletion in | Breast, etc.; |
| | AAA75493.1 | extracellular domain | 4.4-fold higher |
| | | resulted in elevated kinase | expression of HER2- |
| | | activity and | splice in metastatic |
| | | transformation activity) | breast cancer |
| VEGF | P15692 | VEGF165 (deletion of | Tumor metastasis in |
| | GI: 17380528 | exon 6) | lung, colon, and |
| | | | glioblastoma; macular |
| | | | degeneration |
| | | VEGF121 (deletion of | Breast cancer, lung, |
| | | exon 6&7) | pancreatic beta cell |
| | | | carcinogenesis, colon |
| | | | and melanoma |
| MDM2 | BT007258 | 7 short isoforms from | Breast cancer |
| | AAP35922.1 | exon deletions, correlate | |
| | | with high grade | |
| | | malignancy | |
| FGF-8 | D38752 | Isoform FGF-8a, -8b, -8e, | Breast cancer |
| | BAA22527.1 | or -8f; | |
| | | FGF-8b isoform over- | |
| | | expressions correlate with | |
| | | cancer | |
| PSM | NM_004476 | PSM″ (226 nucleotide | Prostate cancer - |
| | NP_004467.1 | deletion) | High level in carcinoma |
| | | | cells, basal level in |
| | | | normal tissue |
| PSA | BC005307 | Deletion amino acid | Prostate cancer |
| | AAH05307.1 | 45-88; several other | |
| | | spliced variants were | |
| | | found in cancer | |
| KLK2 | BC005196 | Deletion exon 4 | Prostate cancer |
| | AAH05196.1 | | |
| Insulin R | NM_000208 | Exon17 deletion | Diabetes |
| | NP_0001999.1 | (truncated IR, no tyrosine | |
| | | kinase domain) | |
| Enovin (PDNF) | AJ245628 | Tissue specific spliced, | Neuronal disgeneration |
| | CAB52396.1 | and diseased/spliced form | |
| (TCR)-zeta | J04132 | Codon insert between | lupus erythematosus |
| | AAA60394.1 | exonIV/V, deletions of | and other disease |
| | | exon II, VI, VII, (V + VI), | indications |
| | | (VI + VII), (II + III + IV), | |
| | | (V + VI + VII) | |
| KST1 | NM_062944 | Exon 6 deletion | BFIC, ICCA Syndroms |
| (sodium/glucose | NP_443176.2 | | |
| transporter) | | | |
| Dystrophin | S73125 | Several isoforms from | muscular dystrophy |
| | AAB20696.1 | exon deletions | |
| IL-6 receptor | NM_000565 | IL6R-soluble | RA |
| | NP_000556.1 | | |
| CD44 | NM_000610 | CD44R1 (V8-10) | Colon, Lung |
| | | CD44v7/8 | Rheumatoid arthritis |
| | | | (only in diseased joins) |
| | | CD44v6 | Breast cancer |
| | | CD44v5 | Wilms tumor; |
| | | | increased expression |
| | | | correlate with tumor |
| | | | stage |
| | | CD44v7 | Autoimmune, bowel |
| | | CD44v3 | Autoimmune, skin |
| TGF -alpha | NM_003236 | Variant I (VaI) | Increased transforming |
| | | | activity in several tumor |
| | | | lines |
| | | Variant II (VaII) | Increased transforming |
| | | | activity in several tumor |
| | | | lines |
| tenascin-C | NM_002160 | Deletion FNIII domain | bullous keratopathy |
| | | | human corneas |
| Troponin T2 | NM_000364.1 | Deletion Exon 7 | Cardiomyopathy |
| | | (12 amino acid | |
| Troponin I | NM_000363 | Soluble form in serum | Cardiomyopathy |

| Viral Disease | Virus Protein | GenBank Accession No. |
|---|---|---|
| SARS | Spike coat protein | GI: 30027620 |
| HIV | HIV-1 (env) | AY223790 |
| | | AAP57385.1 |
| HCV, Hepatitis C virus | polyprotein | NC_004102 |
| | | NP_67149.1 |

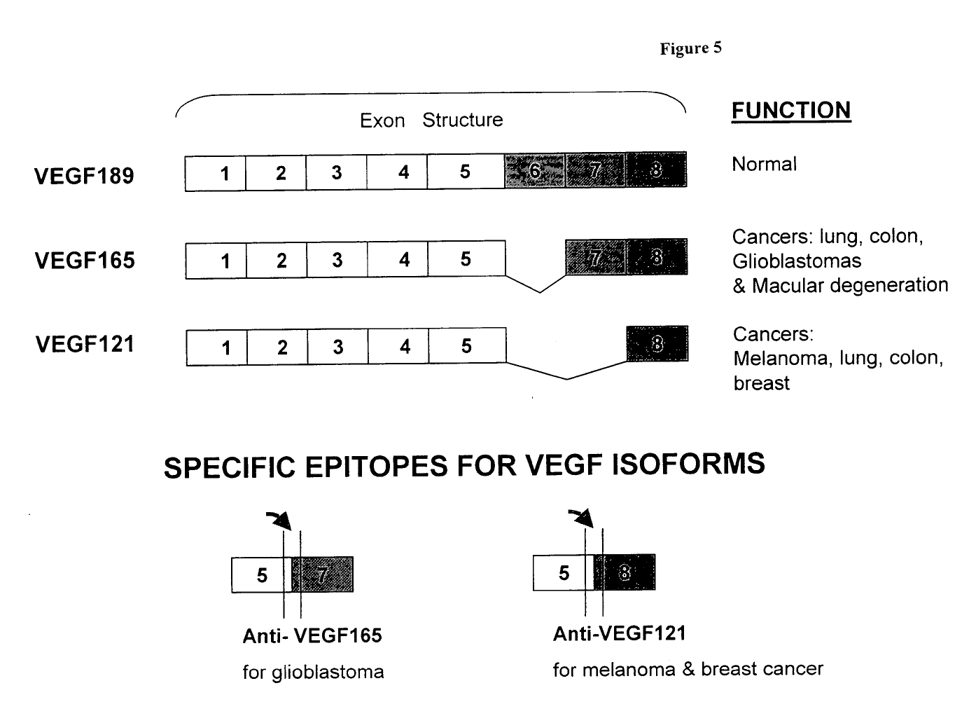

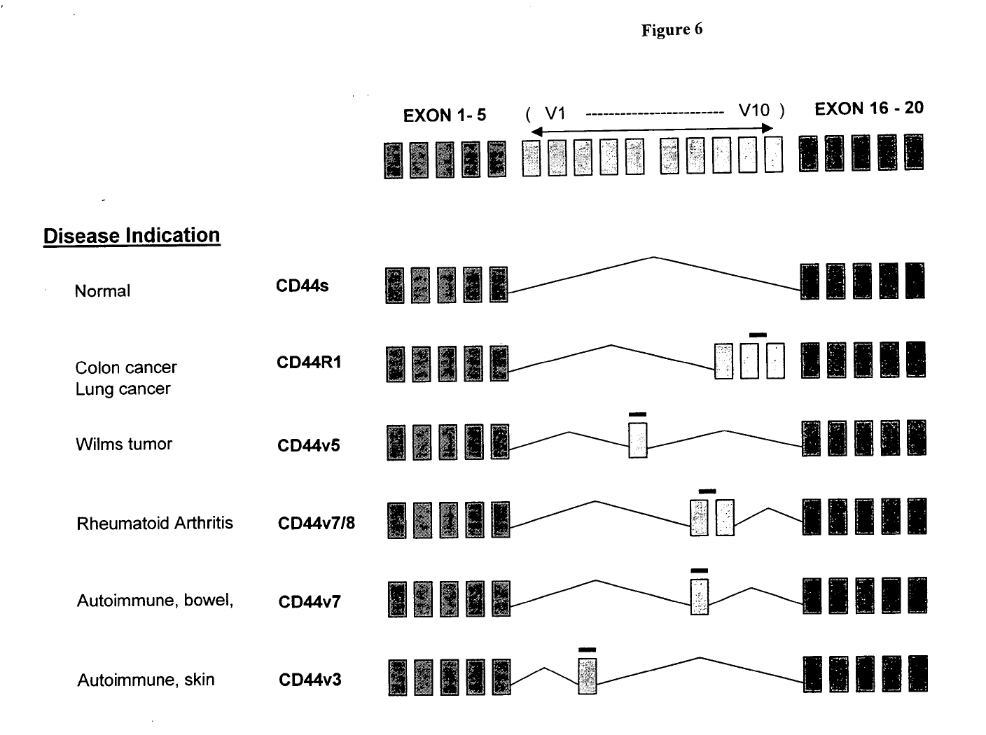

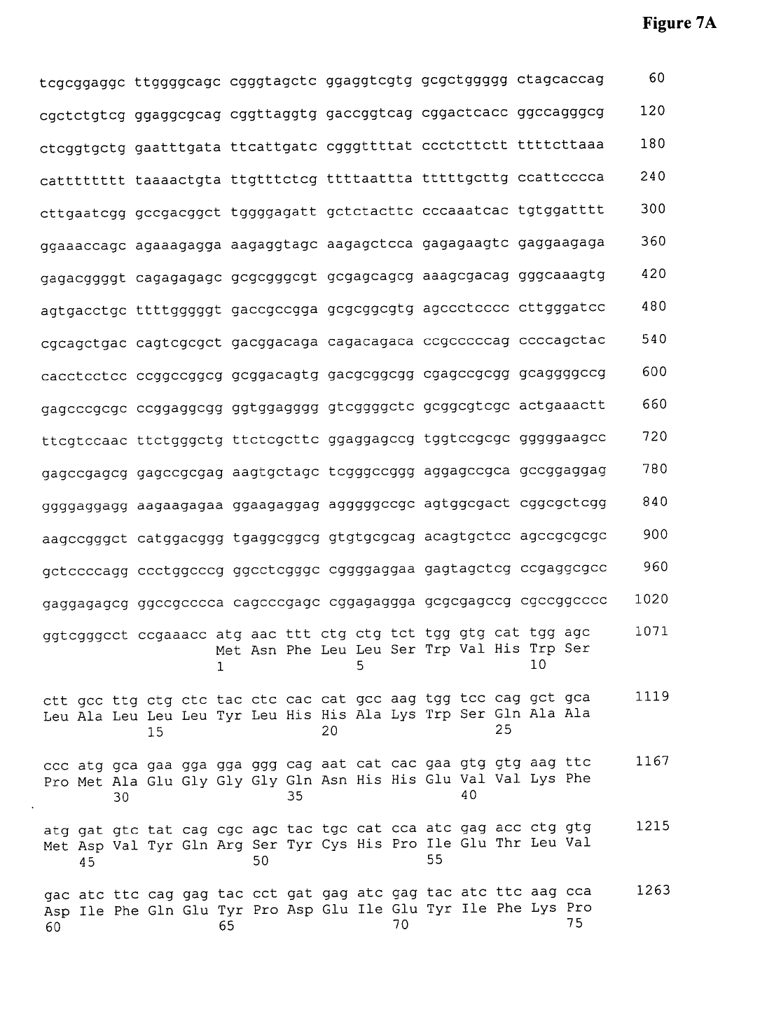

Examples of diseases that are associated with different protein isoforms include but are not limited to rheumatoid arthritis, diabetes, acute myeloid leukaemia (AML), chronic lymphocytic leukaemia (CLL), ovarian cancer, prostate cancer, breast cancer, colorectal cancer, glioblastoma, melanoma, lung cancer, renal carcinoma, muscular dystrophy, neuropsychiatric disorder, autosomal dominant polycystic kidney disease (ADPKD), cardiovascular disease, Alzheimer's disease. Protein isoforms associated with cancers of the lung and colon include vascular eptithelial growth factor (VEGF)165 and VEGF121 (see Examples and FIG. 5). Isoforms associated with cancers of the bladder, breast, ovary and lung include HER2 isoform 1 and HER2 isoform 2 (see Examples). Isoforms associated with various cancers and autoimmune diseases also include CD44 isoforms CD44R1, CD44v5, CD44v7/8, CD44v7, and CD44v3 (see Examples and FIG. 6).

Therapeutic antibodies may also target G protein coupled receptors (GPCRs). Indeed, 60% of currently marketed drugs target various GPCRs, and there are currently no effective ways to raise antibodies to these receptors. Accordingly, antibodies to short sequences located in the extracellular domain of these receptors can be prepared as described herein.

Examples of pathogenic diseases and proteins that can be targeted by antibodies include infections with bacteria, viruses, microplasma and parasites. Viruses include influenza viruses, human immunodeficiency viruses (HIV), hepatitis viruses, such as Hepatitis C viruses, and coronaviruses, such as Severe Acute Respiratory Syndrome (SARS-CoV) coronavirus, tubercle bacilillus that causes tuberculosis (TB) and Plasmodium that causes malaria.

Antibodies conjugated to a label that is capable of producing a detectable signal or to other functional moieties, such as toxins are also provided herein. When fused to a toxin, a drug or a pro-drug, an antibody may be referred to as an “immunotoxins”. Antibodies generated by methods described herein may be chemically bonded to a toxin or label by any of a variety of well-known chemical procedures. For example, when the label or cytotoxic agent is a protein and the second component is an antibody, the linkage may be by way of heterobifunctional cross-linkers, e.g., SPDP, carbodiimide glutaraldehyde, or the like or by recombinant methods.

In one embodiment, conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See W094/11026.

The labels may be covalently linked to the antibodies, or conjugated through a secondary reagent, such as a second antibody, protein A, or a biotin-avidin complex. Suitable labels include, but are not limited to, radionuclides, enzymes, substrates, cofactors, inhibitors, fluorescent dyes, chemiluminescent dyes, bioluminescent compounds and magnetic particles. See, for examples of patents teaching the use of such labels, U.S. Pat. Nos. 3,817,837; 3,850,752; 3,939,350; 3,996,345; 4,277,437; 4,275,149; and 4,366,241. Labels can be used to track or irradiate cells in a subject.

Immunotoxins, including single chain molecules, may also be produced by recombinant means. Production of various immunotoxins is well-known in the art, and methods can be found, for example in “Monoclonal Antibody-Toxin Conjugates: Aiming the Magic Bullet,” Thorpe et al. (1982) Monoclonal Antibodies in Clinical Medicine, Academic Press, pp. 168-190; Vitatta, Science (1987) 238:1098-1104; and Winter and Milstein (1991), Nature 349:293-299.

A variety of cytotoxic agents are suitable for use in immunotoxins. Cytotoxic agents include, but are not limited to, radionuclides, such as Iodine-131, Yttrium-90, Rhenium-188, and Bismuth-212; a number of chemotherapeutic drugs, such as vindesine, methotrexate, adriamycin, and cisplatinum; and cytotoxic proteins such as ribosomal inhibiting proteins like pokeweed antiviral protein, Pseudomonas exotoxin A, ricin, diphtheria toxin, ricin A chain, etc., or an agent active at the cell surface, such as the phospholipase enzymes (e.g., phospholipase C). See, generally, “Chimeric Toxins,” Olsnes and Phil Pharmac. Ther., 15:355-381 (1981); and “Monoclonal Antibodies for Cancer Detection and Therapy,” eds. Baldwin and Byers, pp. 159-179, 224-266, Academic Press (1985).

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). Antibodies may also be conjugated to a prodrug-activating enzyme which converts a prodrug (e.g. a peptidyl chemotherapeutic agent, see W081/01145) to an active anti-cancer drug. See, for example, WO 88/07378 and U.S. Pat. No. 4,975,278. Accordingly, the enzyme component of the immunoconjugate may include an enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as .beta.-galactosidase and neuraminidase useful for converting glycosylated prodrugs into free drugs; .beta.-lactamase useful for converting drugs derivatized with .beta.-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert prodrugs into free active drugs [see, e.g., Massey, Nature 328: 457-458 (1987)]. Antibody-abzyme conjugates can be prepared as described herein for delivery of the abzyme to a tumor cell population.

Enzymes can be covalently bound to the antibody by techniques well known in the art such as the use of heterobifunctional crosslinking reagents. Alternatively, enzyme/antibody fusion comprising at least the antigen-binding region of an antibody linked to at least a functionally active portion of an enzyme of the invention can be constructed using recombinant DNA techniques well known in the art [see, e.g., Neuberger et al., Nature, 312: 604-608 (1984)].

Antibodies can be administered to a subject in the form of a pharmaceutical composition comprising a therapeutically effective amount of antibody and a pharmaceutically acceptable carrier (additive) and/or diluent. For example, in the case of solid tumors, compositions comprising antibodies may be injected into, or in the vicinity of, the tumor. In the case of a cancer of a blood cell, e.g., leukemia, compositions may be administered into the blood or the bone marrow. The effective amount of antibody to administer may depend on the particular disease to be treated, the stage of the disease and the age of the patient.

Pharmaceutical compositions suitable for parenteral administration may comprise one or more antibodies in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the antibody from subcutaneous or intramuscular injection. This may be accomplished by the use of a liquid suspension of crystalline or amorphous material having poor water solubility. The rate of absorption of the antibody then depends upon its rate of dissolution which, in turn, may depend upon crystal size and crystalline form. Alternatively, delayed absorption of a parenterally-administered antibody is accomplished by dissolving or suspending the antibody in an oil vehicle.

Injectable depot forms may be made by forming microencapsule matrices of the antibodies in biodegradable polymers such as polylactide-polyglycolide. Depending on the ratio of antibody to polymer, and the nature of the particular polymer employed, the rate of antibody release can be controlled. Examples of other biodegradable polymers include poly(orthoesters) and poly(anhydrides). Depot injectable formulations are also prepared by entrapping the antibody in liposomes or microemulsions which are compatible with body tissue.

The methods described herein, in particular the epitope scanning methods, can also be used to identify peptides and nucleic acids encoding such for use in vaccination. In one embodiment, a DNA vaccine against a disease, such as cancer or a pathogenic disease, is generated based on the results of an epitope scanning of the gene encoding a target protein of the disease. For example, various peptides covering the whole target protein fused to a carrier protein, or nucleic acids encoding such, are administered to a host animal for eliciting immune response in the body. The immunogenecities of the epitope peptides can be determined by testing the anti-serum of the immunized animals. Fusion proteins carrying epitope peptides, or a fragment of or a full length protein of the targeted isoform can be used to measure the titer of the antiserum using the assay method described above. Vaccines can then be designed based on these results. In particular, vaccines may comprise peptides or nucleic acids encoding such, to which antibodies have been produced in the host animal. Specifically, high titer of the antiserum towards a peptide indicates that the peptide is particularly immunogenic. Several epitopes with high immunogenicity either from a single target protein isoform or several protein isoforms may be selected and used in combination in a vaccine regiment for a given disease.

To test the efficacy of a DNA vaccine, the vaccine may be given to an experimental animal model. Animal models are well known in the art for numerous diseases, for example, for human tumors. In an illustrative embodiment, a vaccinated animal will be challenged with inoculated human tumors either before or after vaccination with a DNA vaccine. A protective or positive effect of the vaccine should be reflected by reduced tumor burden in the experimental animals. Without wanting to be limited to a particular mechanism of action, a tumor-specific vaccine may stimulate either one or both body's immune arms, i.e. cellular immunity and humoral immunity.

Also provided herein are methods for preparing vaccines, such as DNA vaccines, against a disease, comprising (i) identifying one or more epitopes of a protein associated with the disease and (ii) preparing peptides comprising an amino acid sequences of one or more epitopes or including nucleotide sequences encoding one or more epitopes into an expression vector. Methods for identifying epitopes on a target protein are further described herein. “Disease associated proteins” or “proteins associated with a disease” refer to proteins that can be targeted for treating or preventing a disease.

4. Diagnostic Uses

Antibodies may further be used in diagnostic assays for detecting antigens e.g., in specific cells, tissues, or bodily fluids, such as serum. In one embodiment, a biological sample is obtained from a subject having, e.g., cancer, and the presence of one or more cancer associated isoforms of a protein are tested for. The presence of an isoform that is associated with cancer would indicate that the subject has or is likely to develop cancer. Similarly, antibodies can be used to detect the presence of pathogens in a subject or in any tissue or cell or sample in vitro.

Diagnostic methods may comprise using two or more antibodies, where, e.g., one antibody that is specific to a disease-associated protein, is not of sufficient specificity for a clear diagnosis. The two or more antibodies can be applied simultaneously or sequentially to the sample to be tested.

The same antibodies described in the above “therapeutic” section can be used in diagnostic assays.

Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²p, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982 ).

Also provided are methods for determining the presence of an antigen in a sample, e.g., a biological sample such as a bodily fluid or a sample of cells or tissue. A method may comprise (i) contacting a sample with a solid surface comprising a plurality of antibodies located at specific locations on the solid surface under conditions in which antigen/antibody complexes form specifically; (ii) further contacting the solid surface with a plurality of fusion proteins, wherein each fusion protein comprises a polypeptide that binds specifically to an antibody on the solid surface and a carrier protein, under conditions in which antigen/antibody complexes form specifically; and (iii) detecting the presence of the carrier protein at each specific location on the solid surface, wherein the absence of the carrier protein at a specific location indicates the presence of antigen binding specifically to the antibody located at the specific location, thereby indicating the presence of the antigen in the sample. The solid surface may be an antibody array, which can be obtained commercially or prepared according to methods known in the art. The solid surface may comprise at least about 10; 100; 1000; 10,000; or 100,000 antibodies. A person of skill in the art will recognize that other molecules can be used in the place of antibodies, provided that the molecules bind specifically to proteins. An exemplary method is shown in FIG. 3. The solid surface may comprise a plurality of antibodies binding specifically to one antigen, or to different antigens. The antigens may be disease-associated antigens, such as cancer-associated or pathogenic organism associated antigens.

Antibodies also are useful for the affinity purification of antigen from recombinant cell culture or natural sources. In this process, antibodies may be immobilized on a suitable support, such as Sephadex resin or filter paper, using methods well known in the art. The immobilized antibody may then be contacted with a sample containing the antigen to be purified, and thereafter the support is washed with a suitable solvent that will remove substantially all the material in the sample except the antigen, which is bound to the immobilized antibody. Finally, the support is washed with another suitable solvent that will release the antigen from the antibody.

5. Kits

The present invention provides kits, such as diagnostic and therapeutic kits, as well as kits for preparing and/or screening antibodies. For example, a kit may comprise one or more pharmaceutical composition as described herein and optionally instructions for their use. Kits may also comprise one or more devices for accomplishing administration of such compositions. For example, a subject kit may comprise a pharmaceutical composition and catheter for accomplishing direct intraarterial injection of the composition into a cancerous tumor. In other embodiments, a subject kit may comprise pre-filled ampoules of a protein isoform specific antibody construct, optionally formulated as a pharmaceutical, or lyophilized, for use with a delivery device.

Kits may comprise a container with a label. Suitable containers include, for example, bottles, vials, and test tubes. The containers may be formed from a variety of materials such as glass or plastic. The container may hold a composition which includes an antibody that is effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Preparation of Monoclonal Antibodies Binding Specifically to Disease Associated Isoforms of Vascular Endothelial Growth Factor (VEGF)

Vascular endothelial growth factor (VEGF) has been identified as one of the most important factors mediating angiogenesis in physiological and pathological conditions. The human VEGF gene consists of 8 exons corresponding to the following nucleotides of the cDNA set forth in GenBank Accession Number P15692 .

Human VEGF Coding Sequences:

| | NT | |
|---|---|---|
| Exon 1: | 1-66 | 23 aa |
| Exon 2: | 67-108 | 14 aa |
| Exon 3: | 109-315 | 69 aa |
| Exon 4: | 316-392 | 25 aa |
| Exon 5: | 393-422 | 10 aa |
| Exon 6: | 423-542 | 40 aa |
| Exon 7: | 543-674 | 44 aa |
| Exon 8: | 675-692 | 6 aa |
| Total | | 231 aa |
| Signal peptide | | 26 aa |

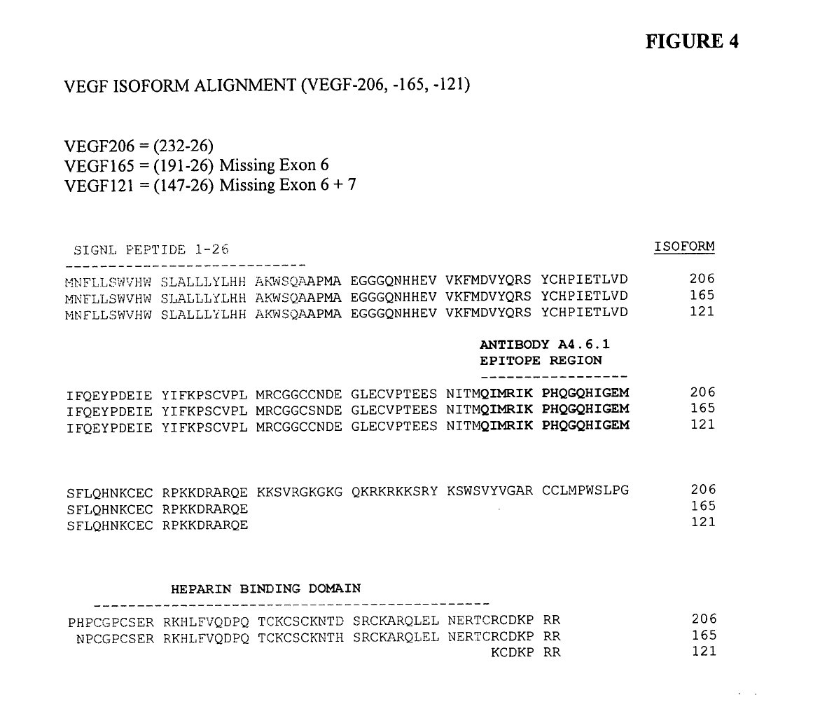

Through alternative splicing, four isoforms of VEGF are formed, consisting of 206, 189, 165, and 121 amino acids, and referred to as VEGF206, VEGF189, VEGF165 and VEGF121, respectively. An alignment of forms 206, 165 and 121 is shown in FIG. 4. The same 115 N-terminal residues of VEGF are shared by all four of these isoforms. VEGF206 and VEGF189 differ from VEGF165 and VEGF121 in their bioavailability, with the longer forms (VEGF206 and VEGF189) being matrix-bound and the shorter forms being freely diffusible. Sites of expression of these forms varies: VEGF165 and VEGF121 are significantly upregulated in cancers of lung and colon; VEGF189 is expressed in normal lungs; and VEGF206, a precursor, is hardly detectable in any tissues (Houck K A, Ferrara N, Winer J, Cachianes G, Li B, Leung D W., Mol Endocrinol. 1991 Dec.; 5(12):1806-14)). The nucleotide sequence of VEGF165 and 121 are provided in GenBank Accession numbers AAM03108/AF486837_{—}1 and AAF19659/AF214570_{—}1.

Monoclonal antibodies against the two isoforms that are expressed in cancer tissues will be prepared as follows (see FIG. 5). The following nucleotide sequences from each of the two isoforms will be inserted in frame with a nucleotide sequence encoding mouse SEAP into an expression vector: for VEGF-165 specific antibodies: 5′ cta tct cgt tct gtt ctt tta ggg aca ccc gga acg agt ctc 3′ (SEQ ID NO: 1) encoding the following amino acid sequence: DRARQE/NPCGPCSE (SEQ ID NO: 2); FOR VEGF-121 specific antibodies: 5′ cta tct cgt tct gtt ctt ttt aca ctg ttc ggc tcc gcc 3′ (SEQ ID NO: 3) encoding the following amino acid sequence: DRARQEKCDKPRR (SEQ ID NO: 4) where “/” represents the junctional site between 2 exons. The fusions will be either peptide-SEAP or SEAP-peptide. Variants of these sequences can also be used, e.g., sequences that encompass an exon junction but differ in one or more amino acids from the sequences set forth here, in particular at the N- and C-termini. The amino acid sequence of the isoforms is set forth in FIG. 4, and any sequence encompassing an exon junction can be used, e.g., sequences comprising 3, 5, 7, 10, or 15 amino acids at one end or the other of the junction.

The vectors are then introduced into mice according to standard procedures. Alternatively proteins consisting of the VEGF peptides linked to SEAP, which can be made in COS cells, are administered to mice. The anti-serum titers will be monitored at one to two week intervals after immunization. Animals with high titer will be used for isolation of spleenocytes. Preparation of hybridomas using spleenocytes and myeloma cells will be performed according to standard procedures.

Antibodies in the culture supernatant of hybridoma cells will be tested for antigen binding using a high throughput ELISA protocol. Accordingly, the supernatant of the hybridomas will be transferred from 96-well culture plates into 96-well or 384-well assay plates that are pre-coated with goat anti-mouse IgG (or rabbit anti-mouse). In the 384-well format, 5 to 10 microliter of culture medium will be used per assay. After incubation at room temperature for 30 minutes, assay plates will be washed to eliminate unbound antibodies. SEAP-epitope fusion proteins will then be added into the wells and incubated for 30 minutes at room temperature. Unbound SEAP-epitope fusions will be washed away. Antigen-antibody binding will be detected by addition of alkaline phosphate substrate and measured on a plate reader. A high SEAP activity will indicate the presence of antibody recognizing the VEGF epitope.

Anti-VEGF165 and -121 antibodies will be validated with standard immunochemistry assays, such as Western blotting using recombinant proteins of VEGF-165 and VEGF-121. Anti-VEGF165 is expected to bind specifically to VEGF165, but not to other isoforms of VEGF proteins such as VEGF-121 and full length VEGF206. Anti-VEGF121 is expected to bind specifically to VEGF121, but not to other isoforms of VEGF proteins.

These antibodies can be used to test protein samples prepared from cultured tumor cell lines, such as non-small lung tumor myeloma, and frozen tumor tissue slices, e.g., by immuno-histochemistry on tumor tissue slides. The biological activity of the antibodies can be tested in mitogenesis assays on endothelial cells following the previously described procedure (Hiratska et al. Proc Natl. Acad. Sci. U.S.A. 95:9349-54 (1998), Shibuya et al. Curr Top In Micro & Immu. 237:59-83 (1999)). Anti-VEGF165 or -121 antibodies that have neutralizing activities should block VEGF mediated function on endothelial cells.

### Example 2

### Preparation of DNA Vaccines Comprising Disease Associated Isoforms of Vascular Endothelial Growth Factor (VEGF)

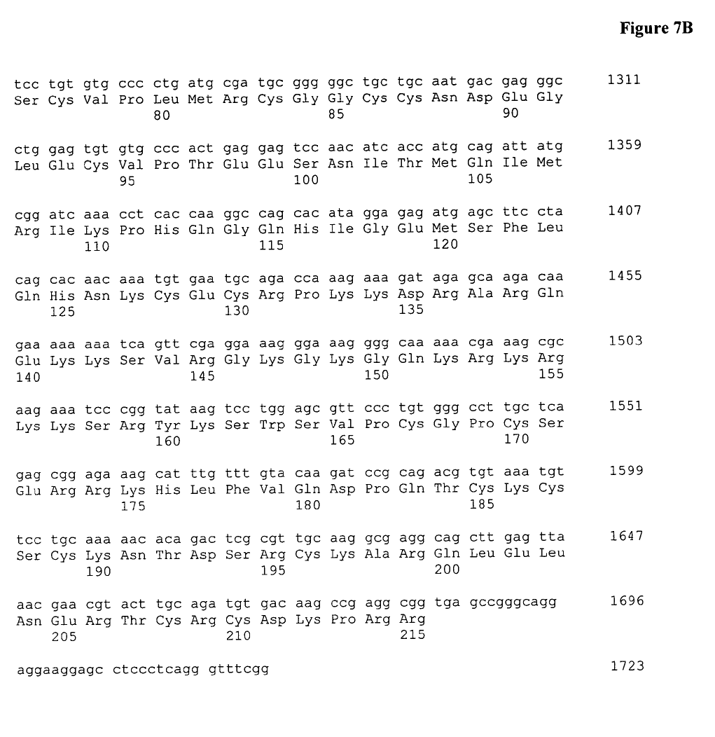

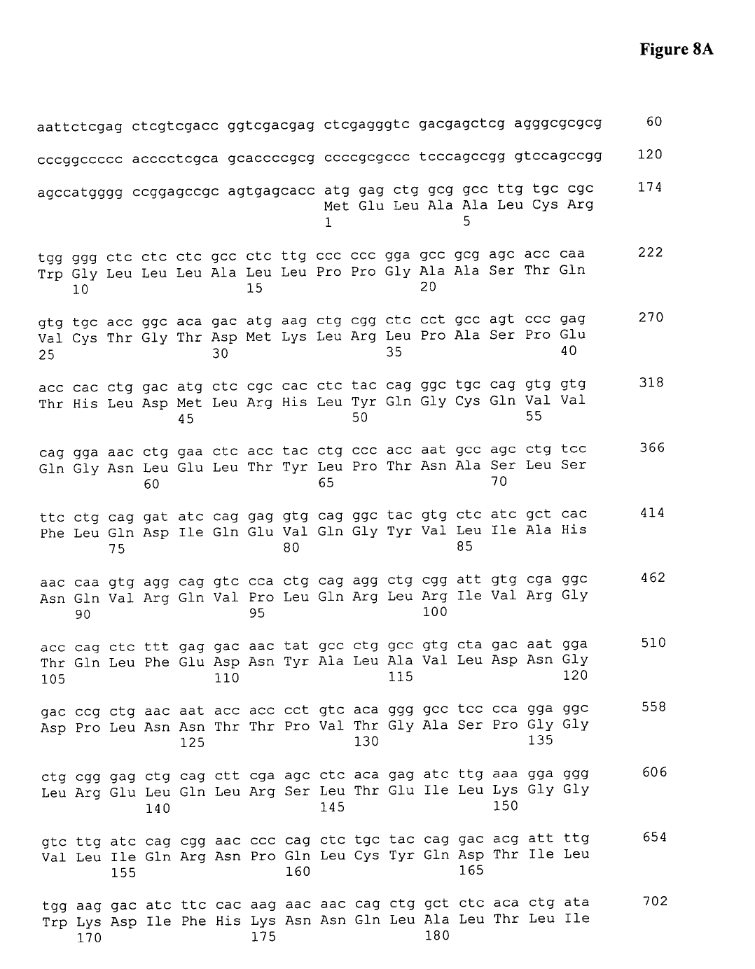

DNA vaccines targeting to VEGF165 and VEGF121 may be developed in experimental animals with epitope specific sequences as indicated above. Essentially, oligonucleotides encoding specific epitopes will be inserted into an expression vector for production of secreted peptides in vivo. The expression vector may contain a coding sequence for a secreted protein as a carrier protein that facilitates the expression and/or secretion of the epitope peptides. The choice of carrier protein may be a serum albumin or other secretary peptides, or a cytokine. A DNA vaccine for a given disease, for example colon cancer, may consist of epitope sequences from VEGF-165, the oligonucleotide: 5′ gat aga gca aga caa gaa aat ccc tgt ggg cct tgc tca gag 3′ (SEQ ID NO: 1) encoding the following amino acid sequence: DRARQE/NPCGPCSE (SEQ ID NO: 2); and from VEGF-121, the oligonucleotide: 5′ gat aga gca aga caa gaa aaa tgt gac aag ccg agg cgg 3′ (SEQ ID NO: 3) encoding the following amino acid sequence: DRARQE/KCDKPRR (SEQ ID NO: 4) where “/” represents the junctional site between 2 exons. Variants of these sequences can also be used, e.g., sequences that encode contiguous amino acids forming an exon junction but differ in one or more nucleotides from the sequences set forth here, in particular at the 5′ and 3′ ends. The nucleotide sequence of human VEGF is set forth as SEQ ID NO: 14 (GenBank Accession No. NM_{—}003376) and FIG. 7, and any sequence encoding contiguous amino acids encompassing an exon junction can be used, e.g., sequences comprising 10, 20, 30 or 50 nucleotides at one end or the other of the junction.

The DNA vaccine to cancer related VEGF isoforms will be tested in animal models for angiogenesis inhibitors as previously described. Particularly, these anti-VEGF isoform vaccines will be tested with given human tumors that were demonstrated for the involvements of either or both VEGF165 and 121 isoform with the metastasis of the tumor. Ideally, these anti-VEGF isoform vaccines can be used for cancer patients with early stages of diagnosed cancers, who can benefit from prevention of tumor spreading by blocking the activities of angiogenesis factors, such as VEGF 165 and or VEGF 121.

### Example 3

### Preparation of Monoclonal Antibodies Binding Specifically to Disease Associated Isoforms of ErbB-2

A number of anti-ErbB-2 (mouse protein) or anti-Her2 (human protein) mAbs have been isolated, and one such Ab, 4D5 or Herceptin, has demonstrated efficacy in the treatment of metastatic breast cancer (Schaller et al. J. Cancer Res. Clin. Oncol. 125:520 (1999) and Shak et al. Herceptin Multinational Investigator Group. Semin Oncol. 26:71 (1999)). However, since c-erbB-2 also play physiological functions in other tissues, such as heart, the side effect of antibodies to HER2 included heart failures, which caused a number of cases of death (Horton et al. Cancer Control. 9(6):499-507 (2002). The epitope of 4D5 is reported to be within amino acid 529-627 of the extracellular domain (ECD) (Sliwkowski et al. Semin Oncol. 26:60 (1999). The nucleotide sequence of Her2 (or HER2) is provided in GenBank Accession number P04626.

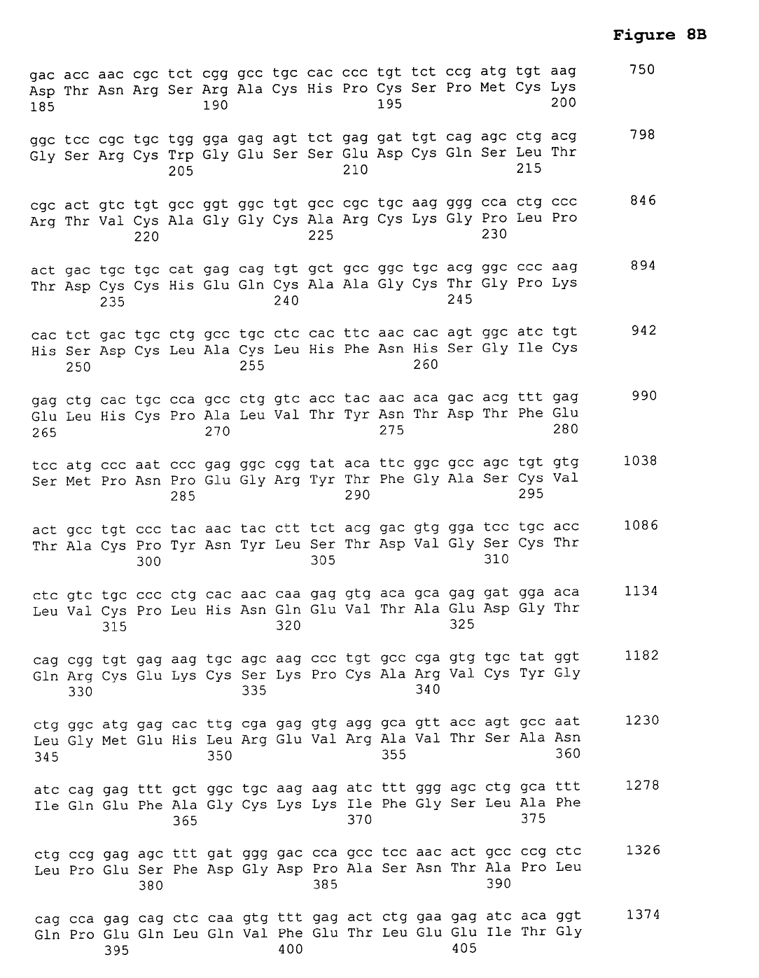

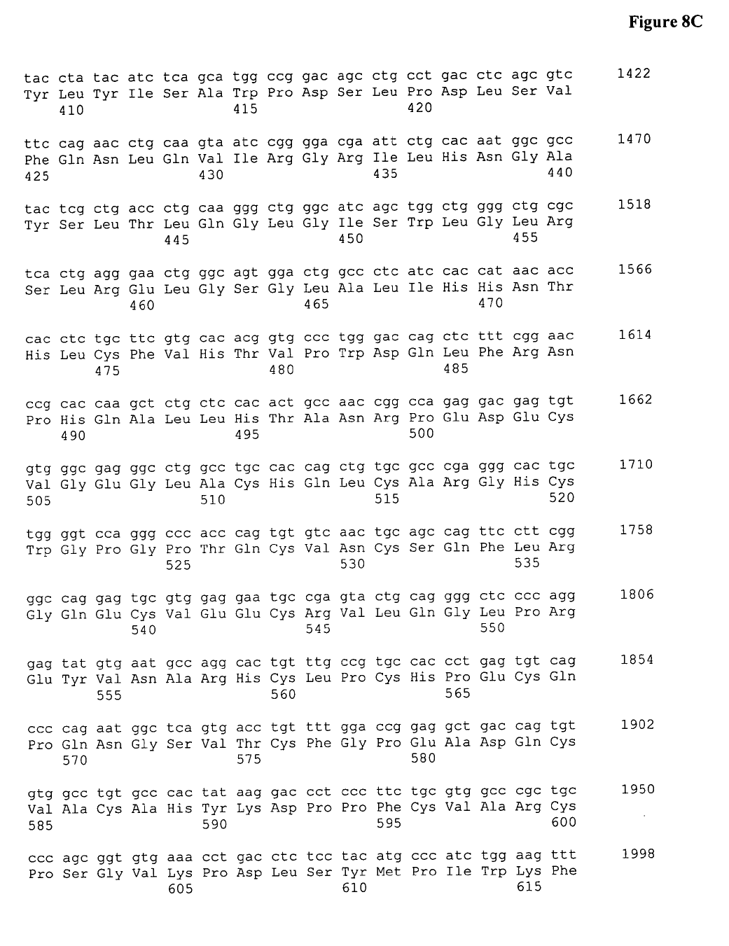

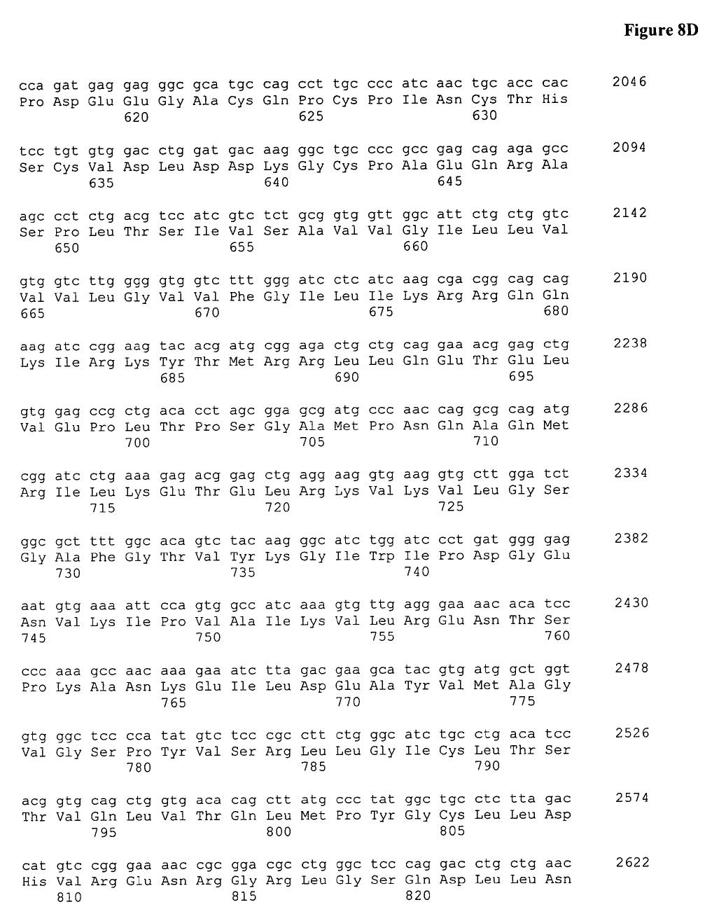

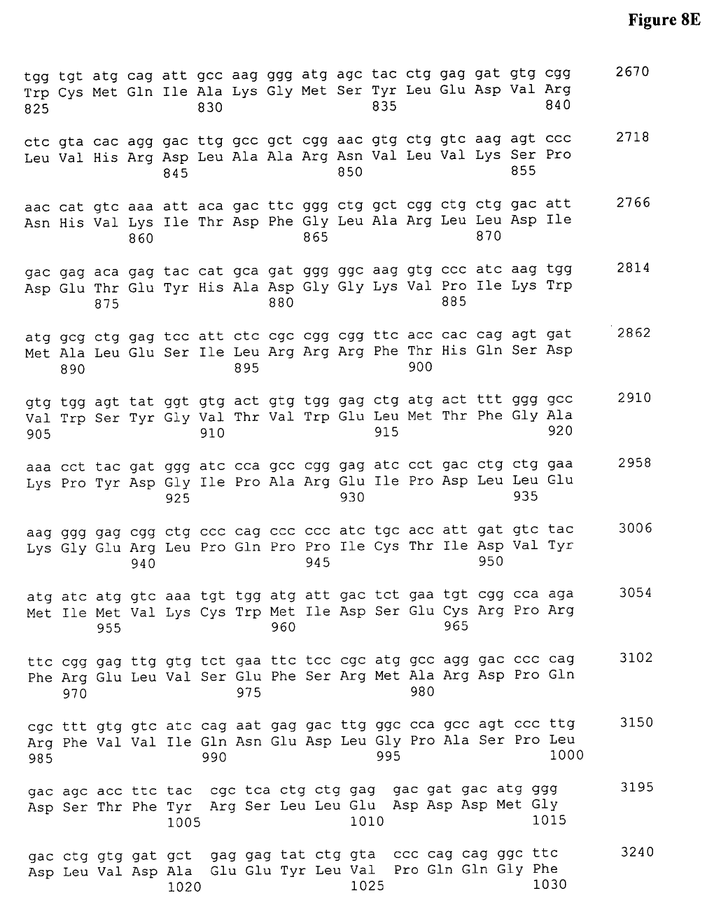

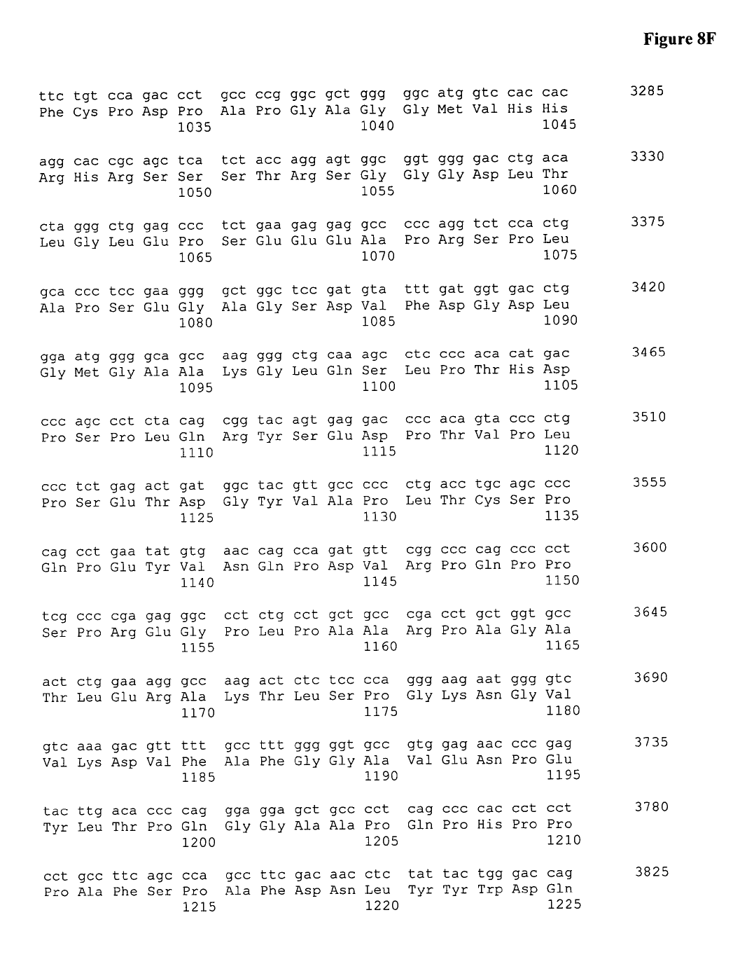

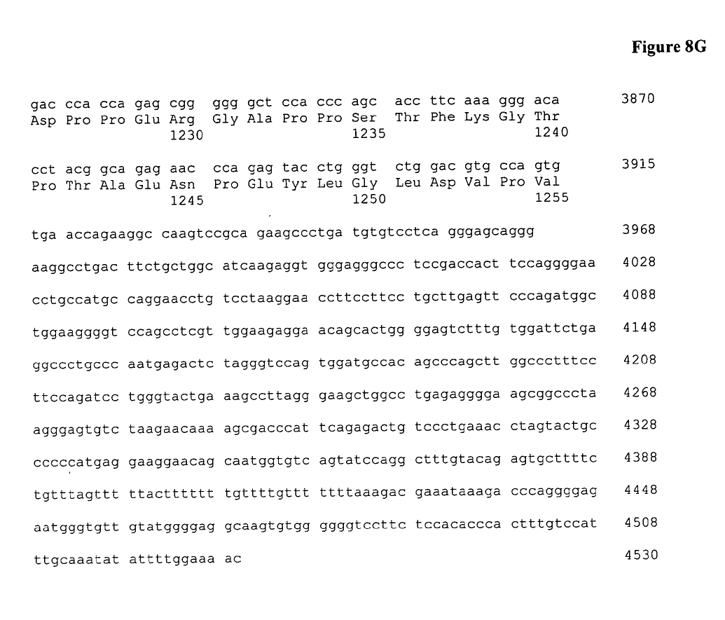

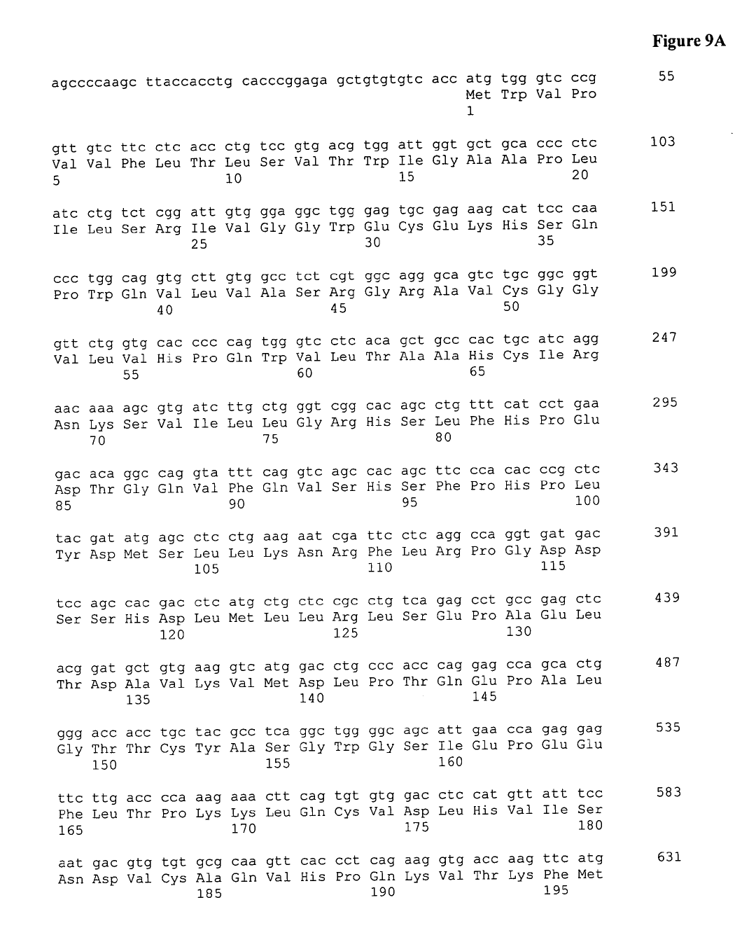

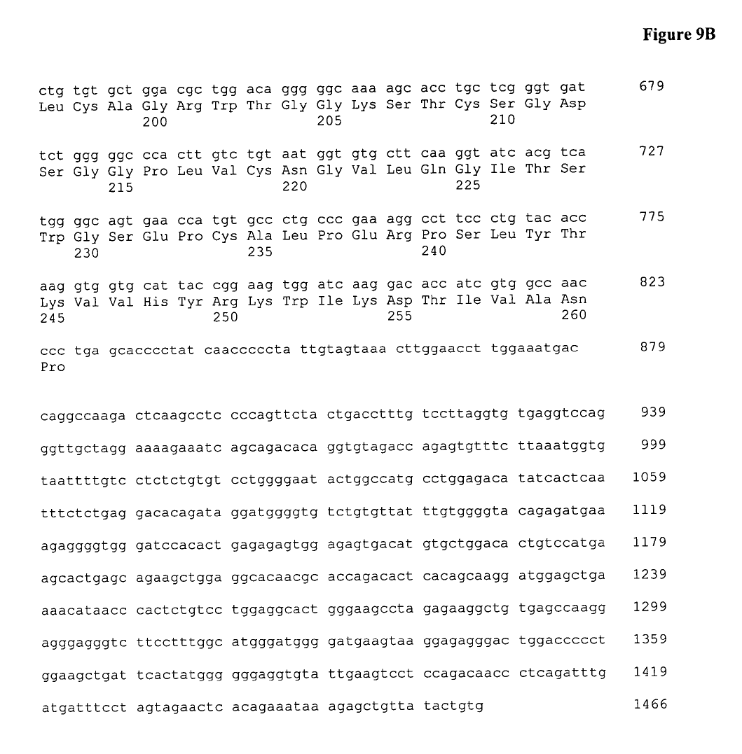

Monoclonal antibodies to the disease associated isoforms of Her2 will be prepared as follows. To target the specific isoform HER2-splice variant having a deletion of 16 amino acids in the ECD (amino acids 634 to 649) (“splice” or “HER2 splice isoform 1”), the following sequence will be used as a peptide: INCTHS/PLTS (SEQ ID NO: 6) (“/” represents an exon junction). To target the specific isoform of HER2 (ECD DEL) that is missing 12 amino acids in the ECD (amino acids 636 to 647) (“ECD DEL” or “HER2 splice isoform 2”), the following sequences will be used as a peptide: CTHSCV/ASPLT (SEQ ID NO: 8) (“/” represents exon junction). Variants of these sequences can also be used, e.g., sequences that encompass an exon junction but differ in one or more amino acids from the sequences set forth here, in particular at the N- and C-termini. The amino acid sequence of human HER2 is set forth in SEQ ID NO: 17 and in FIG. 8, and any sequence encompassing an exon junction can be used, e.g., sequences comprising 3, 5, 7, 10, or 15 amino acids at one end or the other of the junction.

Monoclonal antibodies will be obtained as described above for the VEGF antibodies either by hybridoma technology or by phage display technology.

Antibodies to the HER2 peptides will be tested for specificity for HER2 isoforms: antibodies are expected to bind to the two isoforms to which they were raised, but not to the wild type or other isoform of HER2. Anti-HER2 (splice) and anti-HER2 (ECD DEL) will be tested on tumor tissue slides from breast cancer and on cells of non-small cell lung cancers. These cancer cells express HER2 (splice) and/or HER2 (ECD DEL), and antibodies should detect a positive signal by immuno-histochemistry tests. Normal tissues such as heart tissue should not express these variant isoforms of HER2. The neutralizing activity of isoform specific antibodies of HER2 can be tested in animal models following previously described procedures. (Schaller et al. J. Cancer Res. Clin. Oncol. 125:520 (1999).

### Example 4

### Preparation of DNA Vaccines to Disease Associated Isoforms of ErbB-2

DNA vaccines targeting to HER2 (splice) and HER2 (ECD DEL) may be developed for therapeutics and prevention of breast tumor and ovarian tumor similarly as indicated above for anti-VEGF isoform vaccines. DNA vectors comprising the following nucleotide sequences will be prepared: 5′ atc aac tgc acc cac tcc/cct ctg acg tcc 3′ (SEQ ID NO: 5) (HER2 splice) and 5′ tgc acc cac tcc tgt gtg/gcc agc cct ctg acg 3′ (SEQ ID NO: 7) (HER2 ECD DEL). Variants of these sequences can also be used, e.g., sequences that encode contiguous amino acids forming an exon junction but differ in one or more nucleotides from the sequences set forth here, in particular at the 5′ and 3′ ends. The nucleotide sequence of human HER2 is set forth as SEQ ID NO: 16 (GenBank Accession No. NM_{—}004448) and FIG. 8, and any sequence encoding contiguous amino acids encompassing an exon junction can be used, e.g., sequences comprising 10, 20, 30 or 50 nucleotides at one end or the other of the junction.

The vaccines will be tested in known animal models.

### Example 5

### Preparation of Monoclonal Antibodies Binding Specifically to Prostate Cancer Associated Isoform of Prostate Specific Antigen (PSA)

PSA, encoded by the hKLK3 gene, is well known as the most powerful tool to diagnose and monitor patients with prostate cancer. However, its weak point has become apparent from a numerous reports [see, e.g. Stamey, T. A., N. Eng. J. Med., 317:909-917 (1987); Arai,); Arai, J. Urol., 144:1415-1419 (1990); Catalona, W. J., Eng. J. Med., 324:1156-1161 (1987); Heuze-Vourc'h, N., Eur J Biochem 268(16):4408-13 (2001); Tanaka, T., Cancer Res. 60(1):56-9 (2000); Heuze-Vourc'h, N., Eur J Biochem 270(4):706-14 (2003)]. Best characterized as a differential antigen, PSA is not a cancer-specific protein. PSA is present in the serum as a mixture of several molecular species. Differential splicing of hKLK3 gene contributes to the molecular heterogeneity of free-PSA in the serum of patients with benign or malignant prostate tumors. Certain spliced forms showed tight correlation with prostate cancer (see, e.g. Tanaka, T., Cancer Res. 60(1):56-9 (2000); Heuze-Vourc'h, N., Eur J Biochem 270(4):706-14 (2003)). These molecular species of PSA should be used for better diagnostic products and therapeutic drugs.

Monoclonal antibodies recognizing a specific PSA isoform, PSA-delta44 will be prepared as follows. This PSA isoform is a result of alternative splicing, which leads to deletion of 44 amino acid residues (amino acid 45-88) from mature PSA. The epitope design for isoform PSA-delta44 is the following. To target the isoform of PSA-delta44, the sequence spanning the junction site of the deletion will be used as a peptide: AHCIR/RPGDD (SEQ ID NO: 10) or HCIR/KPGDDS (SEQ ID NO: 11) (“/” represents the junction site). The peptide will be conjugated to a carrier protein. The conjugated peptide will be used as an immunogen for producing monoclonal antibodies by hybridoma technology, or by phage display technology.

Additionally, the nucleotide encoding the above mentioned amino acid sequence will be synthesized: 5′ gcc cac tgc atc agg/agg cca ggt gat gac 3′ (SEQ ID NO: 9). The synthetic oligos will be ligated into expression vectors for production of a fusion protein carrying PSA-delta44 epitope peptide and a carrier protein. Such an expression vector expressing a fusion protein, i.e. peptide (HCIR/KPGDDS)-SEAP can be used to immunize mouse for production of hybridomas. Monoclonal antibodies specific to PSA-delta44 will be screened according to positive binding to immunogen peptide (HCIR/KPGDDS)-SEAP. SEAP readouts will provide positive detection of PSA-delta44 epitope binding by antibodies.

A person of skill in the art will recognize the variants of these sequences can be used, based, e.g., on the known nucleotide and amino acid sequences of human PSA: GenBank accession No. X05332 for human mRNA for PSA precursor and CAA28947 for protein for PSA precursor. These sequences are set forth as SEQ ID Nos: 18 and 19, respectively.

Isoform specific antibody to PSA will be validated with serum samples obtained from patients with prostate cancer. Serum samples from healthy group will be used as negative controls.
Normal PSA (Underlining Indicates the Region that is Absent in the PSA-delta44.) 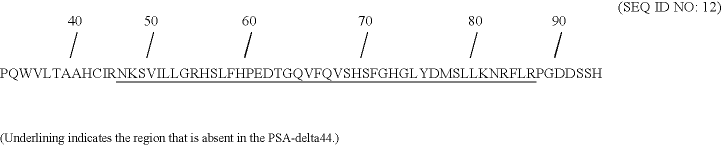
PSA-delta44 (Bold Letters Indicate the Sequence as an Epitope for Antibody to PSA-delta44. “/” between Letters Indicates the Junction Site after the Deletion.)

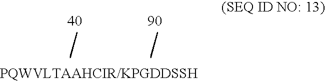

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. I. Freshney, ed. (1987)), Manipulating the Mouse Embryo, (Cold Spring Harbor Laboratory Press, Cold Spring Harbor, N.Y., 1986).

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for identifying an antibody to a target protein from a plurality of antibodies, comprising
i. providing antibodies, wherein at least one antibody binds specifically to a fusion protein comprising at least a portion of a target protein linked to a carrier protein;
ii. linking at least some of the antibodies to a solid surface to obtain a solid surface coated with antibodies, wherein different antibodies are located on different areas of the solid surface;
iii. contacting the solid surface coated with antibodies with the fusion protein; and
iv. conducting an assay to determine the presence of the carrier protein, wherein the presence of a carrier protein indicates the presence of an antibody to the target protein.

2. The method of claim 1, wherein the target protein comprises an isoform of a protein or a portion thereof sufficient for raising an antibody against it.

3. The method of claim 2, wherein the isoform of the protein is an isoform of a protein that is associated with a disease.

4. The method of claim 1, wherein the target protein comprises a viral protein or a portion thereof sufficient for raising an antibody against it.

5. The method of claim 1, wherein the carrier protein comprises secretory alkaline phosphatase (SEAP) or a portion thereof sufficient for enzymatic activity.

6. The method of claim 1, wherein the carrier protein comprises horseradish peroxidase or a portion thereof sufficient for enzymatic activity.

7. The method of claim 1, wherein the carrier protein comprises beta-galactosidase or a portion thereof sufficient for enzymatic activity.

8. The method of claim 1, wherein the carrier protein comprises luciferase or a portion thereof sufficient for enzymatic activity.

9. The method of claim 1, wherein the carrier protein comprises IgG Fc (gamma chain).

10. The method of claim 1, wherein the antibodies are linked to a solid surface coprising Protein A Sepharose.

11. The method of claim 1, wherein the antibodies are linked to a solid surface comprising Protein G Sepharose.

12. The method of claim 1, wherein the assay used to determine the presence of the carrier protein is a chemiluminescence assay.

13. The method of claim 1, wherein the assay used to determine the presence of the carrier protein is a fluorescence assay.

14. The method of claim 1, wherein the assay used to determine the presence of the carrier protein is a calorimetric assay.

15. The method of claim 1, further comprising a wash step between steps (iii) and (iv) to remove unbound fusion protein.

16. A method for generating a plurality of monoclonal antibodies, wherein each monoclonal antibody binds to a target protein, comprising
i. administering to a host a plurality of fusion proteins or nucleic acids encoding fusion proteins, wherein each fusion protein comprises at least a portion of a target protein and a carrier protein;
ii. preparing a plurality of monoclonal antibody producing cells obtained from cells from the host; and
iii. screening the cells according to the method of claim 1, to obtain a plurality of monoclonal antibodies against the target proteins.

17. The method of claim 16, wherein the target protein comprises an isoform of a protein or a portion thereof sufficient for raising an antibody against it.

18. The method of claim 16, wherein the isoform of the protein is associated with a disease or a portion thereof sufficient for raising an antibody against it.

19. The method of claim 16, wherein the target protein comprises a viral protein or a portion thereof sufficient for raising an antibody against it.

20. The method of claim 16, wherein the carrier protein comprises secretory alkaline phosphatase (SEAP) or a portion thereof sufficient for enzymatic activity.
